(19) United States
(12) Patent Application Publication
(10) Pub. No.: US 2022/0227861 A1
(43) Pub. Date: Jul. 21, 2022

(54) ANTI-MITOTIC COMPOSITION COMPRISING ANTIBODIES AGAINST ZIP6 AND/OR ZIP10

(71) Applicant: University College Cardiff Consultants Limited, Cardiff South Glamorgan (GB)

(72) Inventor: Kathryn Taylor, Cardiff South Glamorgan (GB)

(73) Assignee: University College Cardiff Consultants Limited, Cardiff South Glamorgan (GB)

(21) Appl. No.: 17/618,116

(22) PCT Filed: Jun. 5, 2020
(86) PCT No.: PCT/EP2020/065653

§ 371 Date: Dec. 10, 2021

(30) Foreign Application Priority Data
Jun. 10, 2019 (GB) 1908208.0

(51) Int. Cl. C07K 16/28; A61K 39/395; A61P 35/00

(52) U.S. Cl. CPC C07K 16/28; C07K 2317/76; A61P 35/00; A61K 39/3955

(57) ABSTRACT

The invention relates to an anti-mitotic agent comprising at least one antibody, or an epitope thereof, that selectively binds the extracellular domain of at least one ZIP (Zrt, Irt-like Proteins) transporter protein to inhibit its function, specifically mitosis; a composition comprising same; and the use of said anti-mitotic agent or composition to treat a hyper proliferative disorder including cancer.

### FIELD OF THE INVENTION

The invention relates to an anti-mitotic agent comprising at least one antibody, or a fragment thereof, that selectively binds the extracellular domain of at least one ZIP

(Zrt, Ift-like Proteins) transporter protein to inhibit its function, specifically mitosis; a composition comprising same; and the use of said anti-mitotic agent or composition to treat a hyper proliferative disorder.

### BACKGROUND OF THE INVENTION

Cell proliferation is a prerequisite for any multi-cellular form of life. Cell proliferation, i.e. an increase in cell number starting from a limited number of cells, is thus relevant for any multicellular organism. Cell proliferation is a process which is highly regulated. Apart from the mere increase in biomass of multicellular organisms, multicellular organisms have to control cellular proliferation in order to maintain their highly organized inter-cellular interaction. Any deregulation of cell proliferation represents or results in a pathological condition.

A variety of diseases, including pathological as well as more benign conditions, are characterized by undesirable cell proliferation and in particular increased or hyper proliferation of cells. Many of these disorders are life-shortening, and others drastically reduce the quality of life.

Cancer, for example, is one of the best-known examples of a hyper proliferative disease. Cancer is a large, heterogeneous class of diseases in which a group of cells display uncontrolled growth, resulting in invasion and destruction of adjacent tissues. The cancer cells often metastasize, wherein tumor cells spread to other locations in the body via the lymphatic system or through the bloodstream.

Other forms of hyper proliferative diseases also exist, such as, but not limited to, polycystic kidney disease (PKD) and related cystic kidney diseases, hyperplasia, metaplasia and dysplasia's and their various forms, proliferative disorders of the immune system (such as myelo- and lymphoproliferative disorders), prostatic hypertrophy, endometriosis, psoriasis, tissue repair and wound healing and fibrosis.

Fibrosis involves formation of excess fibrous connective tissue in an organ or tissue and can lead to degeneration of the tissue or organ and/or loss of function if it becomes widespread and aggressive. Further, fibrosis plays a role in a number of disease states in mammals, including, but not limited to, pulmonary fibrosis, idiopathic pulmonary fibrosis, cirrhosis, endomyocardial fibrosis, vascular or spinal stenosis, mediastinal fibrosis, myelofibrosis, retroperitoneal fibrosis, progressive massive fibrosis, nephrogenic systemic fibrosis, Crohn's Disease, keloid or old myocardial infarction, scleroderma/systemic sclerosis, arthrofibrosis, and adhesive capsulitis.

Such hyper proliferative diseases have been known for decades; however, effective treatments remain elusive. Numerous anti-proliferative agents have been reported over the last decade. Many anti-proliferative agents have been investigated, yet few have made significant clinical progress.

Cell proliferative disorders as described herein can occur for various reasons but commonly involve an abnormal mitosis and/or an undesired mitosis.

Zinc is an essential ion in cells; without it, cells cannot sustain life. Zinc is a cofactor for more than 300 enzymes, representing more than 50 different enzyme classes, and is essential for cell growth. Zinc is involved in protein, nucleic acid, carbohydrate, and lipid metabolism, as well as in the control of gene transcription, differentiation and development. Zinc deficiency can be detrimental, causing stunted growth and serious metabolic disorders, while excess zinc can be toxic to cells.

The development of new molecular tools for investigating zinc biology has revealed its multiple biological functions in cells, designating zinc as the ‘calcium of the 21st century’, by demonstrating its signalling diversity. Cellular levels of zinc are tightly regulated by specific zinc transporter proteins, of which there are two known families. These two families have opposing action on zinc transport. The ZnT family (SLC30A) (previously termed CDF for cation diffusion facilitator) of zinc transporters transport zinc out of cells or into intracellular compartments from the cytoplasm, whereas the ZIP family (for Zrt-, Ift-like Proteins) (SLC39A) of zinc transporters transport zinc into the cell cytoplasm from either outside the cell or from intracellular compartments, two of these transporters, ZIP6 (SLC39A6/LIV-1) and ZIP10 (SLC39A10) are close orthologs. Increasing evidence implicates various members of the SLC39A family of ZIP transporters in disease states.

It is herein disclosed that a heterodimer formed by the above two orthologs ZIP6:ZIP10 has a crucial function in initiating mitosis. By targeting specific extracellular ZIP6/ZIP10 domains, we have demonstrated that the inhibition of this heterodimer ZIP6:ZIP10 can completely prevent mitosis and so prevent cell proliferation and growth. This inhibition was observed even in the presence of agents that increase mitosis, and further the inhibition occurred in a dose dependent manner.

Most advantageously, it has also been found that ZIP6 and ZIP10 transporters only translocate to the cell membrane immediately prior to the commencement of mitosis to form a multimeric complex and commence cell division, and thus are only present when a cell is dividing or is rounding to metastasise thereby, elegantly, providing a selective therapeutic target.

Our work therefore provides molecular insight into the long-established role of zinc in proliferation by revealing the key proteins, ZIP6 and ZIP10, in this process and their ability to heterodimerise and so mediate the zinc influx necessary to trigger mitosis. By inhibiting these proteins, we provide a new way of preventing cell division in diseases characterised by cellular hyper proliferation, such as cancer.

### STATEMENTS OF INVENTION

According to a first aspect of the invention there is provided an anti-mitotic composition comprising at least one antibody, or an fragment thereof, that binds the extracellular domain of at least one ZIP (Zrt-, Ift-like Proteins) transporter, wherein said ZIP transporter is either ZIP6 or ZIP10 or a heterodimer comprising both, in order to inhibit its function for use in the treatment of a hyper proliferative disorder by preventing mitosis.

Reference herein to an antibody, or fragment thereof, refers to the part of the antibody that binds the extracellular domain of said zinc transporter, and most ideally prevents the formation of a heterodimer and so/or inhibits the transport of zinc into the cell cytoplasm.

More particularly, said fragment is the complementarity-determining region (CDR) of the antibody or a significant part thereof or an aptamer against a part of the heterodimer, particularly the epitope.

In a preferred embodiment of the invention said composition comprises more than one antibody, each targeting a different part of the ZIP6:ZIP10 heterodimer. Ideally, a combination of antibodies is used, at least one of which binds the ZIP6 transporter part of the heterodimer and at least one of which binds the ZIP10 transporter part of the heterodimer. Additionally, or alternatively, at least one antibody is used that binds both said ZIP6 transporter and said ZIP10 transporter adjacent parts of said heterodimer.

As will be appreciated by those skilled in the art, an antibody can be a polyclonal, or more ideally, a monoclonal antibody, and will be capable of binding a whole or part of the extracellular domain of said transporter.

Ideally an antibody or fragment includes reference to at least the Complementarity Determining Region(s) of said antibody.

Reference herein to the ZIP transporter protein refers to the SLC39A family of zinc transporters that transport zinc into the cell cytoplasm from either outside the cell or from intracellular compartments. As is known to those skilled in the art, the ZIP family contains 14 human sequences, nine of which are in the LIV-1 subfamily, a highly conserved group having eight transmembrane domain proteins that are mainly situated on the plasma membrane and transport zinc into cells. In particular, the human members of the LIV-1 sub family comprise ZIP4, ZIP5, ZIP6, ZIP7, ZIP8, ZIP10, ZIP12, ZIP13, ZIP14.

In a preferred embodiment of the first aspect of the invention, said antibody ideally binds the extracellular domain(s) (ECDs) of said ZIP6 and/or ZIP10 transporter(s), ideally between the following reference co-ordinates which are numbered having regard to the entire N-terminus of the transporter i.e. prior to a cleavage event: N-terminus 1-325 or 1-350 for ZIP6 and 1-407 for ZIP10; yet more preferably between N-terminus 31-325 or 31-344 for ZIP6 and 31-407 for ZIP10. Yet more preferably still, between N terminal amino acids 93-350 for ZIP6 and 46-395 for ZIP10. More ideally still, said antibody binds amino acids 220-350 for ZIP6 and 46-395 for ZIP10. More ideally still, said antibody binds amino acids 246-259 for ZIP6 and 46-59 for ZIP10.

Without wishing to be bound by theory, we believe formation of the heterodimer of ZIP6 with ZIP10 occurs in the endoplasmic reticulum, and N-terminal cleavage of ZIP6 is required for the ZIP6/ZIP10 heterodimer to move to the plasma membrane to initiate cell rounding and import zinc into cells to start mitosis. Through preferential targeting of the extracellular domains of the heterodimer, specifically those of the cleaved N-terminus of ZIP6 and/or the N-terminus of ZIP10 (i.e. those that remain on the proteins after N-terminal cleavage in the ER for translocation), ZIP6 or ZIP10 antibodies can inhibit the ZIP6/ZIP10 heterodimer by binding the N-terminus of one or both transporters and so blocking the zinc influx or interfering with zinc activation, such as protease cleavage, to prevent mitosis.

Most preferably said antibody binds at least one of the following ECDs of ZIP6 between the following reference co-ordinates which are numbered having regard to the entire N-terminus of the transporter i.e. prior to a cleavage event:

N-terminus epitope
(SEQ ID NO: 1 93-HHDHDHHSDHEHHSD-107);
N-terminus epitope
(SEQ ID NO: 2 246-EPRKGFMYSRNTNE-259);
N-terminus and including Transmembrane domain 1
(SEQ ID NO: 3 301-
RSCLIHTSEK KAEIPPKTYS LQIAWVGGFI AISIISFLSL LGVILVPLMN-350);
N-terminus
(SEQ ID NO: 4 303-CLIHTSEKKAEIP-315);
N-terminus
(SEQ ID NO: 5 129-HSHHNHAASGKNKRKALCPDHDSDSSGKDPRNSQGKGAHRPEHASGRRNVKDS
VSASEVTSTVYNTVSEGTHFLETIETPRPGKLFPKDVSSSTPPSVTSKSRVSRLAG
RKTNESVSEPRKGFMYSRNTNENPQE-263);
N-terminus,
(SEQ ID NO: 6 288-NYLCPAIINQIDARSCLIHTSEKKAEIPPKTYSLQIAWVGGFIAISIISF-337);
Extracellular loop TM2-3
(SEQ ID NO: 7 382-ASHHHSHSHEEPAMEMKRGPLFSHLSSQNIEESAYFDSTWK-423);
Extracellular loop TM4-5
(SEQ ID NO: 8 619-TEGLSSG-625);
Extracellular loop TM6-7
(SEQ ID NO: 9 680-HYAENVSM-687);
Extracellular C-terminus
(SEQ ID NO: 10 748-KIVFRINF-755);

Most preferably said antibody binds at least one of the following ECDs of ZIP10 between the following reference co-ordinates which are numbered having regard to the entire N-terminus of the transporter i.e. prior to a cleavage event:

N-terminus,
(SEQ ID NO: 11 46-LEPSKFSKQAAENE-59);
N-terminus
(SEQ ID NO: 12 164-EKETVEVSVKSDDKHMHDHNHRLRHHHRLHHHLDHNTHHFHNDSITPSERGEPS
NEPSTETNKTQEQSDVKLPKGKRKKKGRKSNENSEVITPGFP-253);
N-terminus
(SEQ ID NO: 13 295-QDLDPDNEGELRHTRKREAPHVKNNAIISLR-395);
N-terminus,
(SEQ ID NO: 14 36-LHRQHRGMTELEPSKFSKQAAENEKKYYIEKLFERYGENGRLSFFGLEKL-85);
Extracellular loop TM2-3
(SEQ ID NO: 15 465-GGHDHSHQHAHGHGHSHGHESNKFLEEYDAVLK-497);
Extracellular loop TM4-5
(SEQ ID NO: 16 693-SAGLTGG-699);
Extracellular loop TM6-7
(SEQ ID NO: 17 754-QYANNITLWN-762);
Extracellular loop C-terminus
(SEQ ID NO: 18 824-KIVFDIQF-831).

Yet more preferably still, said antibody is selected from group comprising: ZIP6M, ZIP6Y, ZIP6AM, ZIP6X, ZIP6R, Anti-SLC39A6^{a}, Anti-SLC39A6^{b}, Anti-SLC39A6^{c}, Anti-SLC39A6^{d}, LIV-1/ZIP6 antibody, Anti-SLC39A6^{e}, Anti-SLC39A6^{f}, ZIP62-A, ZIP10, Anti-SLC39A10^{a}, Anti-SLC39A10^{b}, SLC39A10^{a}, Anti-SLC39A10^{c} and SLC39A10^{b} antibody, as shown in Table 1. Most preferably, said antibody, or fragment thereof, is selected from the group comprising: ZIP6Y, ZIP6AM, ZIP10 and Anti-SLC39A10^{a}.

In a further preferred embodiment of the first aspect of the invention, at least two of the afore antibodies, or fragments thereof, are used in combination wherein at least one binds at least a part of the ZIP6 transporter and one binds at least a part of the ZIP10 transporter; alternatively, a single antibody is used that binds the ZIP6:ZIP10 heterodimer in order to inhibit its function.

Reference herein to hyper proliferative disorder refers to any disorder, whether benign or pathological, that is characterised by abnormally high levels of cell proliferation compared with levels that are regarded as acceptable or normal levels for a given cell or tissue type, and would be readily recognised and appreciated by those skilled in the art. Examples of hyper proliferative disorders include, but are not limited to: cancer and various forms thereof such as solid tumours, lymphoma or leukaemia, breast, prostate, colon, brain, lung, pancreatic, gastric, bladder, kidney cancer; melanoma; polycystic kidney disease (PKD) and related cystic kidney diseases; hyperplasia, metaplasia and dysplasia's and their various forms; proliferative disorders of the immune system (such as myelo- and lymphoproliferative disorders); prostatic hypertrophy; endometriosis; psoriasis; tissue repair and aberrant wound healing; and fibrosis such as pulmonary fibrosis, idiopathic pulmonary fibrosis, cirrhosis, endomyocardial fibrosis, vascular or spinal stenosis, mediastinal fibrosis, myelofibrosis, retroperitoneal fibrosis, progressive massive fibrosis, nephrogenic systemic fibrosis, Crohn's Disease, keloid or old myocardial infarction, scleroderma/systemic sclerosis, arthrofibrosis, and adhesive capsulitis, or the like.

In a preferred embodiment of the invention, said hyper proliferative disorder is cancer. Most preferably the cancer referred to herein includes any one or more of the following cancers: nasopharyngeal cancer, synovial cancer, hepatocellular cancer, renal cancer, cancer of connective tissues, melanoma, lung cancer, bowel cancer, colon cancer, rectal cancer, colorectal cancer, brain cancer, throat cancer, oral cancer, liver cancer, bone cancer, pancreatic cancer, choriocarcinoma, gastrinoma, pheochromocytoma, prolactinoma, T-cell leukemia/lymphoma, neuroma, von Hippel-Lindau disease, Zollinger-Ellison syndrome, adrenal cancer, anal cancer, bile duct cancer, bladder cancer, ureter cancer, brain cancer, oligodendroglioma, neuroblastoma, meningioma, spinal cord tumor, bone cancer, osteochondroma, chondrosarcoma, Ewing's sarcoma, cancer of unknown primary site, carcinoid, carcinoid of gastrointestinal tract, fibrosarcoma, breast cancer, Paget's disease, cervical cancer, colorectal cancer, rectal cancer, esophagus cancer, gall bladder cancer, head cancer, eye cancer, neck cancer, kidney cancer, Wilms' tumor, liver cancer, Kaposi's sarcoma, prostate cancer, lung cancer, testicular cancer, Hodgkin's disease, non-Hodgkin's lymphoma, oral cancer, skin cancer, mesothelioma, multiple myeloma, ovarian cancer, endocrine pancreatic cancer, glucagonoma, pancreatic cancer, parathyroid cancer, penis cancer, pituitary cancer, soft tissue sarcoma, retinoblastoma, small intestine cancer, stomach cancer, thymus cancer, thyroid cancer, trophoblastic cancer, hydatidiform mole, uterine cancer, endometrial cancer, vagina cancer, vulva cancer, acoustic neuroma, mycosis fungoides, insulinoma, carcinoid syndrome, somatostatinoma, gum cancer, heart cancer, lip cancer, meninges cancer, mouth cancer, nerve cancer, palate cancer, parotid gland cancer, peritoneum cancer, pharynx cancer, pleural cancer, salivary gland cancer, tongue cancer and tonsil cancer.

In a preferred embodiment said cancer is selected from the group comprising or consisting of solid tumours, lymphoma or leukemia, oesophageal cancer, breast cancer, prostate cancer, renal cell carcinoma, metastatic breast cancer and gastric cancer, colon, brain, lung, pancreatic, gastric, bladder and kidney cancer.

The anti-mitotic agent is particularly useful for the treatment or prevention of those cancers with an unmet clinical need, or those with poor clinical prognosis/outcome. This includes, but is not limited to, chemotherapy resistant cancers, metastatic cancers, currently difficult to treat cancers such as pancreatic and non-small cell lung carcinoma, triple negative breast cancer, hormone-resistant breast cancers, melanomas especially those resistant to B-raf inhibitors.

According to a second aspect of the invention there is provided a pharmaceutical or veterinary composition for use in the treatment of a hyper proliferative disorder, most preferably cancer, comprising the antimitotic agent as defined herein, together with a pharmaceutically or veterinary acceptable excipient or carrier.

Suitable pharmaceutical excipients are well known to those of skill in the art. Pharmaceutical compositions may be formulated for administration by any suitable route, for example oral, rectal, nasal, bronchial (inhaled), topical (including eye drops, buccal and sublingual), vaginal or parenteral (including subcutaneous, intramuscular, intravenous and intradermal) administration and may be prepared by any methods well known in the art of pharmacy.

The composition may be prepared by bringing into association the antibody as defined herein with the carrier. In general, the formulations are prepared by uniformly and intimately bringing into association the antibody with liquid carriers or finely divided solid carriers or both, and then if necessary shaping the product. The invention extends to methods for preparing a pharmaceutical composition comprising bringing the antibody, as defined herein together in conjunction or association with a pharmaceutically or veterinarily acceptable carrier or vehicle.

Formulations for oral administration in the present invention may be presented as: discrete units such as capsules, sachets or tablets each containing a predetermined amount of the active agent; as a powder or granules; as a solution or a suspension of the active agent in an aqueous liquid or a non-aqueous liquid; or as an oil-in-water liquid emulsion or a water in oil liquid emulsion; or as a bolus etc.

For compositions for oral administration (e.g. tablets and capsules), the term “acceptable carrier” includes vehicles such as common excipients e.g. binding agents, for example syrup, acacia, gelatin, sorbitol, tragacanth, polyvinylpyrrolidone (Povidone), methylcellulose, ethylcellulose, sodium carboxymethylcellulose, hydroxypropylmethylcellulose, sucrose and starch; fillers and carriers, for example corn starch, gelatin, lactose, sucrose, microcrystalline cellulose, kaolin, mannitol, dicalcium phosphate, sodium chloride and alginic acid; and lubricants such as magnesium stearate, sodium stearate and other metallic stearates, glycerol stearate, stearic acid, silicone fluid, talc waxes, oils and colloidal silica. Flavouring agents such as peppermint, oil of wintergreen, cherry flavouring and the like can also be used. It may be desirable to add a colouring agent to make the dosage form readily identifiable. Tablets may also be coated by methods well known in the art.

A tablet may be made by compression or moulding, optionally with one or more accessory ingredients. Compressed tablets may be prepared by compressing in a suitable machine the active agent in a free flowing form such as a powder or granules, optionally mixed with a binder, lubricant, inert diluent, preservative, surface-active or dispersing agent. Moulded tablets may be made by moulding in a suitable machine a mixture of the powdered compound moistened with an inert liquid diluent. The tablets may optionally be coated or scored and may be formulated so as to provide slow or controlled release of the active agent.

Other formulations suitable for oral administration include lozenges comprising the active agent in a flavoured base, usually sucrose and acacia or tragacanth; pastilles comprising the active agent in an inert base such as gelatin and glycerin, or sucrose and acacia; and mouthwashes comprising the active agent in a suitable liquid carrier.

Parenteral formulations will generally be sterile.

For topical application to the skin, the composition may be made up into a cream, ointment, jelly, solution or suspension etc. Cream or ointment formulations that may be used for the drug are conventional formulations well known in the art, for example, as described in standard text books of pharmaceutics such as the British Pharmacopoeia.

The precise amount of a composition as defined herein which is therapeutically effective, and the route by which such compound is best administered, is readily determined by one of ordinary skill in the art. Such amounts will depend, of course, on the particular condition being treated, the severity of the condition, the individual patient parameters including age, physical condition, size and weight, the duration of the treatment, the nature of concurrent therapy (if any), the specific route of administration and like factors within the knowledge and expertise of the health practitioner. These factors are well known to those of ordinary skill in the art and can be addressed with no more than routine experimentation. It is generally preferred that a maximum dose of the individual components or combinations thereof be used, that is, the highest safe dose according to sound medical judgment. It will be understood by those of ordinary skill in the art, however, that a patient may insist upon a lower dose or tolerable dose for medical reasons, psychological reasons or for any other reasons. Other factors include the desired period of treatment. In the event that a response in a subject is insufficient at the initial doses applied, higher doses (or effectively higher doses by a different, more localized delivery route) may be employed to the extent that patient tolerance permits.

According to a third aspect of the invention there is provided a combination therapeutic for use in the treatment of a hyper proliferative disorder comprising the above antimitotic agent in combination with at least one other therapeutic agent.

Without limitation, examples of such agents include first-line or adjuvant anti-hormone, radio- or chemo-therapeutics aimed at targeting the primary lesion or suppressing late stage disease progression; for example, anti-HER2 agents such as trastuzumab and pertuzumab and standard adjuvant therapy regimens such as 5-fluorouracil, doxorubicin, and cyclophosphamide (FAC); 5-fluorouracil, epirubicin, and cyclophosphamide (FEC); and doxorubicin and cyclophosphamide (AC); cyclophosphamide, methotrexate, and 5-fluorouracil (CMF); and docetaxel, doxorubicin, cyclophosphamide (TAC). Other suitable agents for use in combination with the compounds of the invention are anti-angiogenic/antimetastatic agents such as bevacizumab (Avastin).

According to a further aspect of the invention there is provided a method of treating a hyper proliferative disorder wherein an antimitotic agent or composition or combination therapeutic according to the invention is administered to a subject having, or suspected of having a hyper proliferative disorder.

In a preferred embodiment of this aspect of the invention, said subject is a mammal. Ideally said mammal is a primate. More ideally, said mammal is human, equine, canine, feline, porcine, ovine, ungulate or any other domestic or agricultural species. Yet most ideally, said mammal is human.

As will be appreciated by those skilled in the art, said method can be utilised in numerous disease context where there is suspected, or diagnosed, hyper proliferative disorder such as, but not limited to, cancer and various forms thereof such as solid tumours, lymphoma or leukaemia breast, prostate, colon, brain, lung, pancreatic, gastric, bladder and kidney cancer; polycystic kidney disease (PKD) and related cystic kidney diseases; melanoma; hyperplasia, metaplasia and dysplasia's and their various form; proliferative disorders of the immune system (such as myelo- and lymphoproliferative disorders); prostatic hypertrophy; endometriosis; psoriasis; tissue repair and aberrant wound healing; and fibrosis such as pulmonary fibrosis, idiopathic pulmonary fibrosis, cirrhosis, endomyocardial fibrosis, vascular or spinal stenosis, mediastinal fibrosis, myelofibrosis, retroperitoneal fibrosis, progressive massive fibrosis, nephrogenic systemic fibrosis, Crohn's Disease, keloid or old myocardial infarction, scleroderma/systemic sclerosis, arthrofibrosis, and adhesive capsulitis, or the like.

In a preferred embodiment of the invention, said hyper proliferative disorder is cancer. Most preferably the cancer referred to herein includes any one or more of the following cancers: nasopharyngeal cancer, synovial cancer, hepatocellular cancer, renal cancer, cancer of connective tissues, melanoma, lung cancer, bowel cancer, colon cancer, rectal cancer, colorectal cancer, brain cancer, throat cancer, oral cancer, liver cancer, bone cancer, pancreatic cancer, choriocarcinoma, gastrinoma, pheochromocytoma, prolactinoma, T-cell leukemia/lymphoma, neuroma, von Hippel-Lindau disease, Zollinger-Ellison syndrome, adrenal cancer, anal cancer, bile duct cancer, bladder cancer, ureter cancer, brain cancer, oligodendroglioma, neuroblastoma, meningioma, spinal cord tumor, bone cancer, osteochondroma, chondrosarcoma, Ewing's sarcoma, cancer of unknown primary site, carcinoid, carcinoid of gastrointestinal tract, fibrosarcoma, breast cancer, Paget's disease, cervical cancer, colorectal cancer, rectal cancer, esophagus cancer, gall bladder cancer, head cancer, eye cancer, neck cancer, kidney cancer, Wilms' tumor, liver cancer, Kaposi's sarcoma, prostate cancer, lung cancer, testicular cancer, Hodgkin's disease, non-Hodgkin's lymphoma, oral cancer, skin cancer, mesothelioma, multiple myeloma, ovarian cancer, endocrine pancreatic cancer, glucagonoma, pancreatic cancer, parathyroid cancer, penis cancer, pituitary cancer, soft tissue sarcoma, retinoblastoma, small intestine cancer, stomach cancer, thymus cancer, thyroid cancer, trophoblastic cancer, hydatidiform mole, uterine cancer, endometrial cancer, vagina cancer, vulva cancer, acoustic neuroma, mycosis fungoides, insulinoma, carcinoid syndrome, somatostatinoma, gum cancer, heart cancer, lip cancer, meninges cancer, mouth cancer, nerve cancer, palate cancer, parotid gland cancer, peritoneum cancer, pharynx cancer, pleural cancer, salivary gland cancer, tongue cancer and tonsil cancer.

In a preferred method of the invention said cancer is selected from the group comprising or consisting of oesophageal cancer, breast cancer, prostate cancer, renal cell carcinoma, metastatic breast cancer and gastric cancer.

The anti-mitotic agent is particularly useful for the treatment or prevention of those cancers with an unmet clinical need, or those with poor clinical prognosis/outcome. This includes, but is not limited to, chemotherapy resistant cancers, metastatic cancers, currently difficult to treat cancers such as pancreatic and non small cell lung carcinoma, triple negative breast cancer, hormone-resistant breast cancers, melanomas especially those resistant to B-raf inhibitors.

According to a further aspect of the invention there is provided an antibody, or fragment thereof, that is specific for ZIP10 wherein said antibody is specific for epitope N-terminus 46-59 amino acids LEPSKFSKQAAENE (SEQ ID NO: 11) of the ZIP10 transporter.

Preferred features of each aspect of the invention may be as described in connection with any of the other aspects.

Throughout the description and claims of this specification, the words “comprise” and “contain” and variations of the words, for example “comprising” and “comprises”, mean “including but not limited to” and do not exclude other moieties, additives, components, integers or steps. Throughout the description and claims of this specification, the singular encompasses the plural unless the context otherwise requires. In particular, where the indefinite article is used, the specification is to be understood as contemplating plurality as well as singularity, unless the context requires otherwise.

All references, including any patent or patent application, cited in this specification are hereby incorporated by reference. No admission is made that any reference constitutes prior art. Further, no admission is made that any of the prior art constitutes part of the common general knowledge in the art.

Other features of the present invention will become apparent from the following examples. Generally speaking, the invention extends to any novel one, or any novel combination, of the features disclosed in this specification (including the accompanying claims and drawings). Thus, features, integers, characteristics, compounds or chemical moieties described in conjunction with a particular aspect, embodiment or example of the invention are to be understood to be applicable to any other aspect, embodiment or example described herein, unless incompatible therewith.

Moreover, unless stated otherwise, any feature disclosed herein may be replaced by an alternative feature serving the same or a similar purpose.

The Invention will now be described by way of example only with reference to the Examples below and to the following Figures wherein:

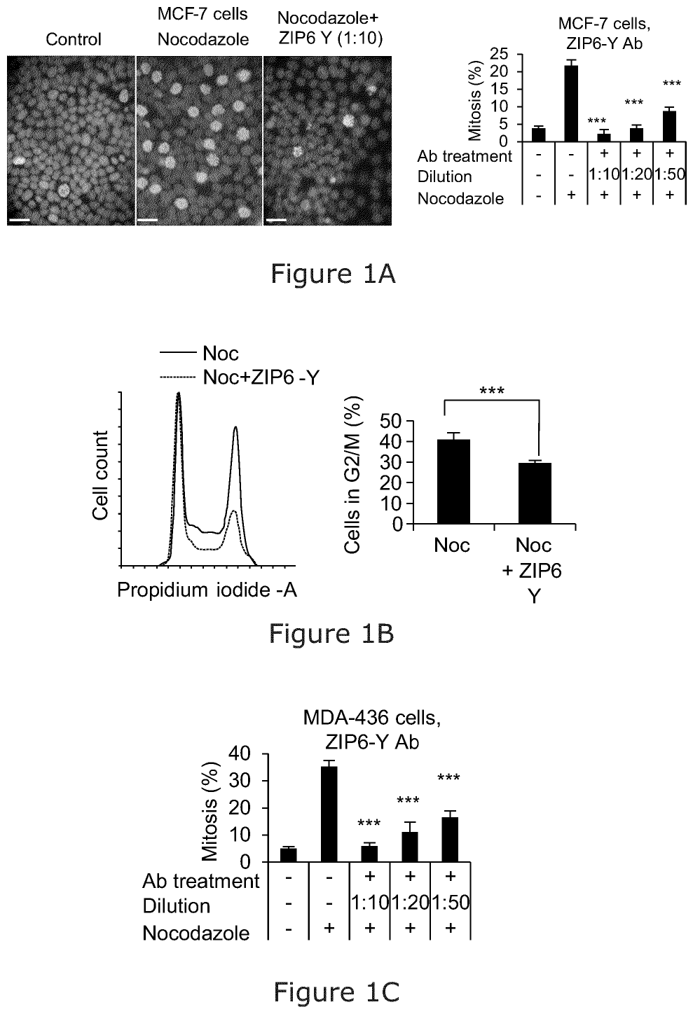

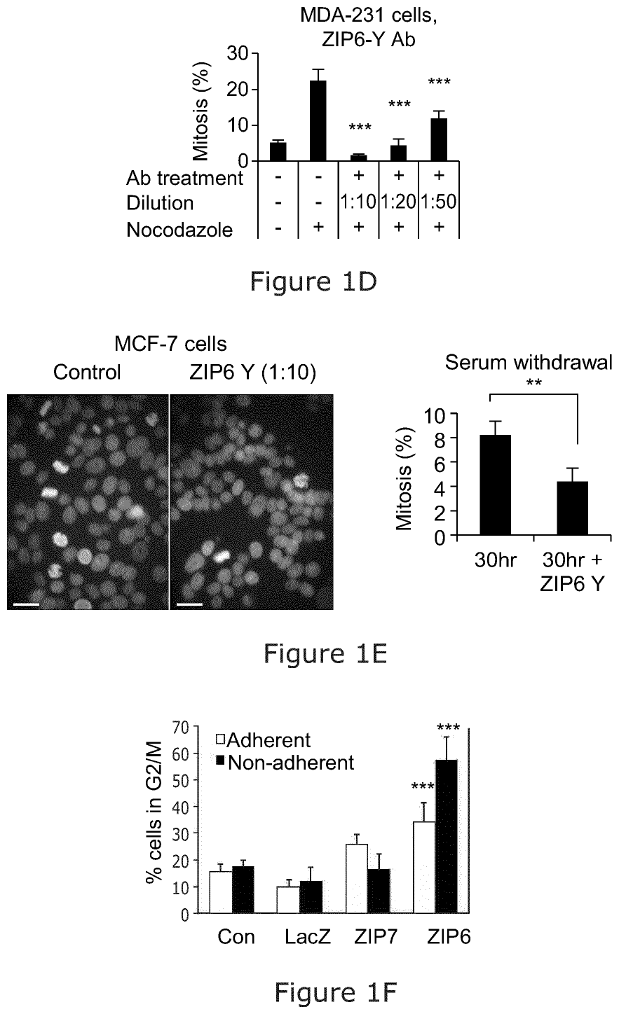

FIG. 1. Shows the requirement of ZIP6 for mitosis. [A] ZIP6-Y antibody treatment with 100 nM nocodazole to synchronize the cell division cycle for 20 hours significantly decreases the number of mitotic cells (positive for pS10HistoneH3, red) in a concentration-dependent manner. [B] FACS cell cycle analysis reveals a significant decrease in the G2/M population in MCF-7 cells treated with ZIP6-Y antibody (1:20). [C-D] Treatment with ZIP6-Y antibody in MDA-436 (C) and MDA-231 cells (D), with 100 nM nocodazole for 20 hours significantly decreases the number of mitotic cells (positive for pS10HistoneH3, red). [E] MCF-7 cells, synchronised by 24-hour serum withdrawal, were fixed 30 hours after serum replacement, the time it takes to enter mitosis after synchronisation. Mitotic count, positivity for pS10HistoneH3 (red), reveals a significant decrease in the number of mitotic cells due to ZIP6-Y antibody treatment (1:10). [F] FACS cell cycle analysis on transfected cells shows a significant increase in G2/M when transfected with ZIP6. Results of at least three independent experiments are demonstrated as mean±SD. For A, C and D, statistical significance is compared to nocodazole-treated samples. ** p<0.01, *** p<0.001. Scale bar, 25 μm.

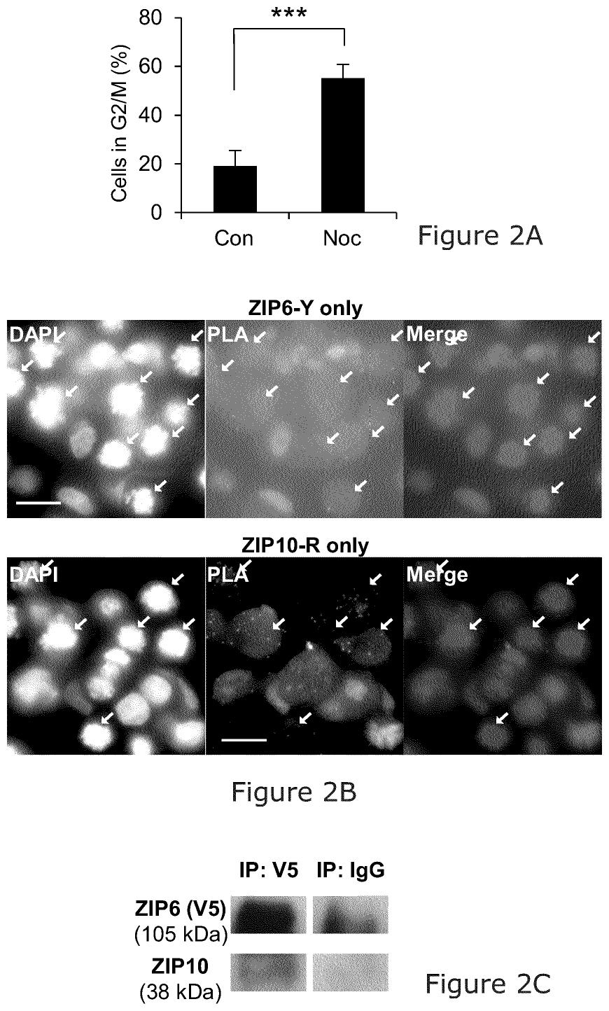

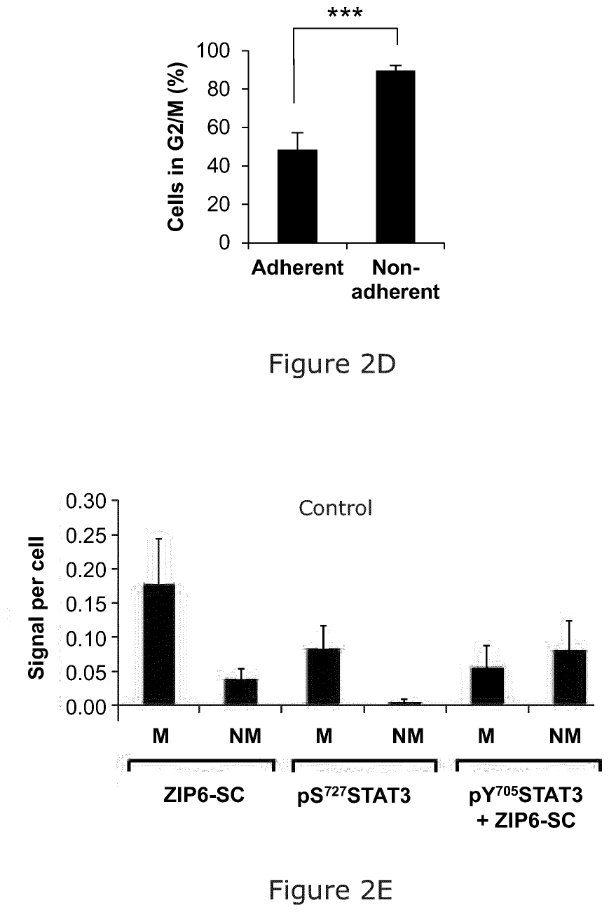

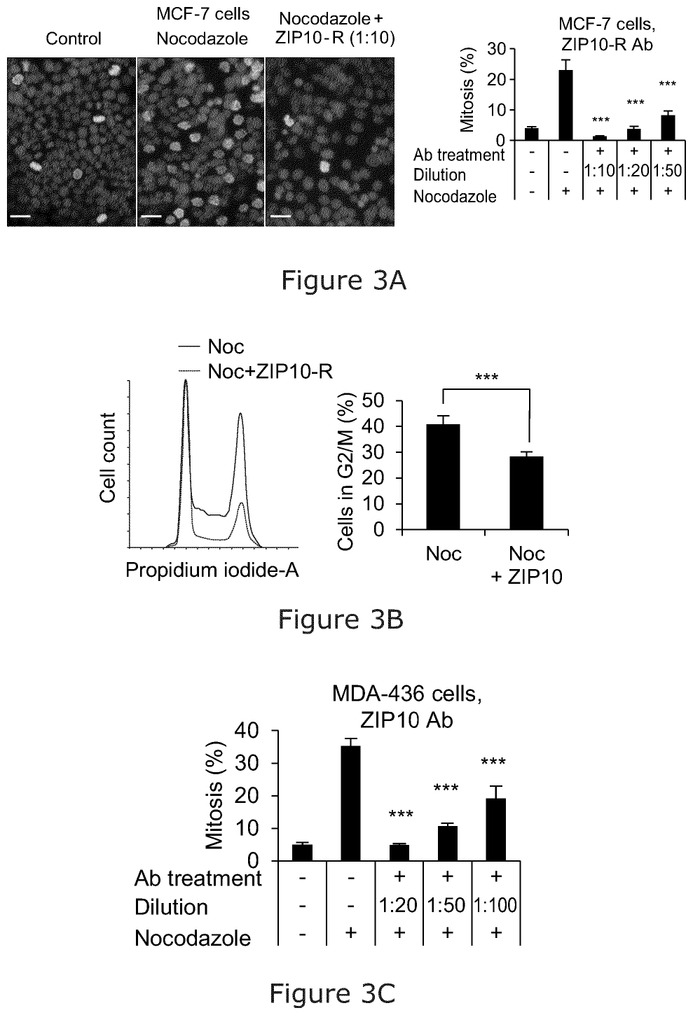

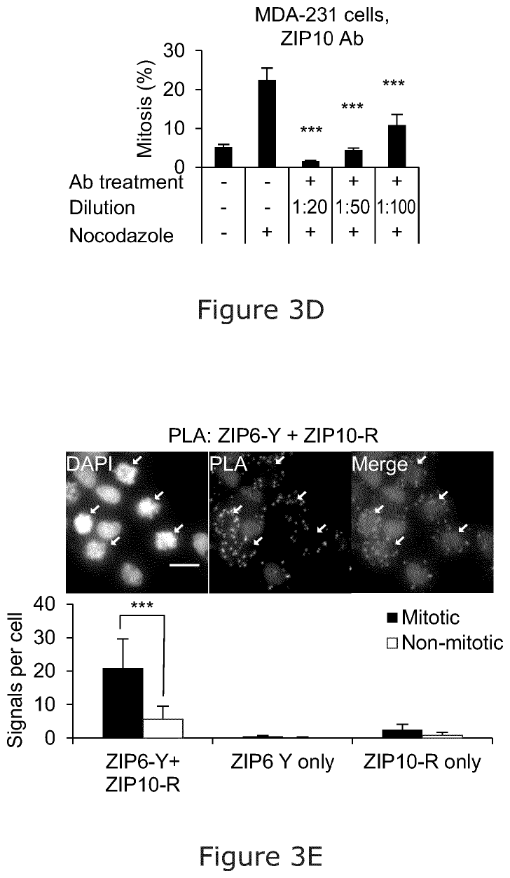

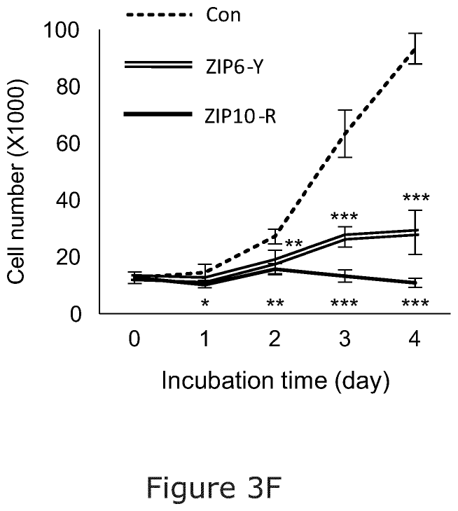

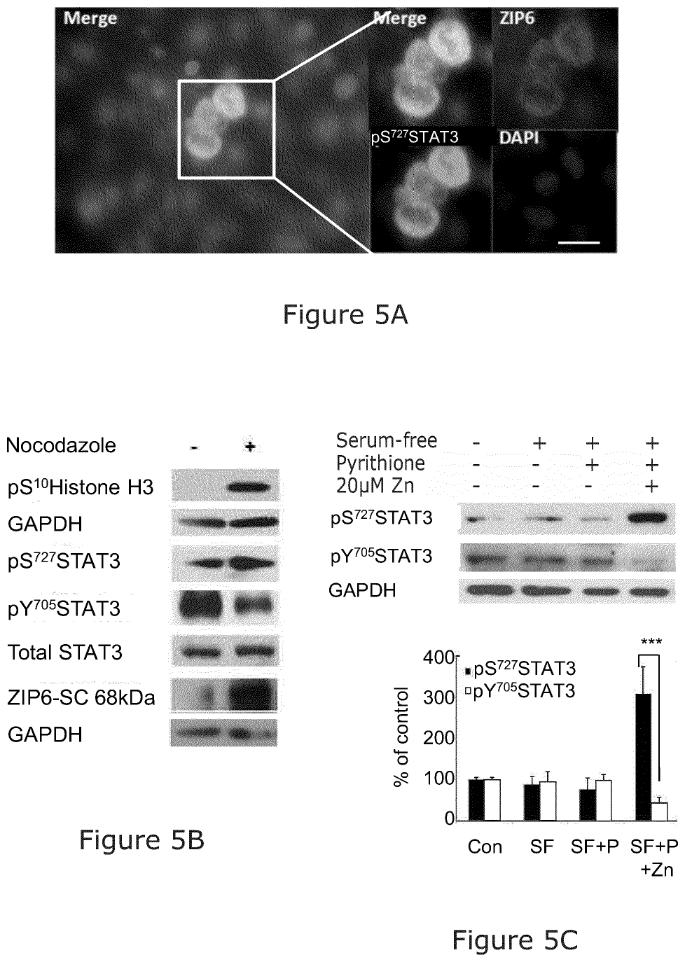

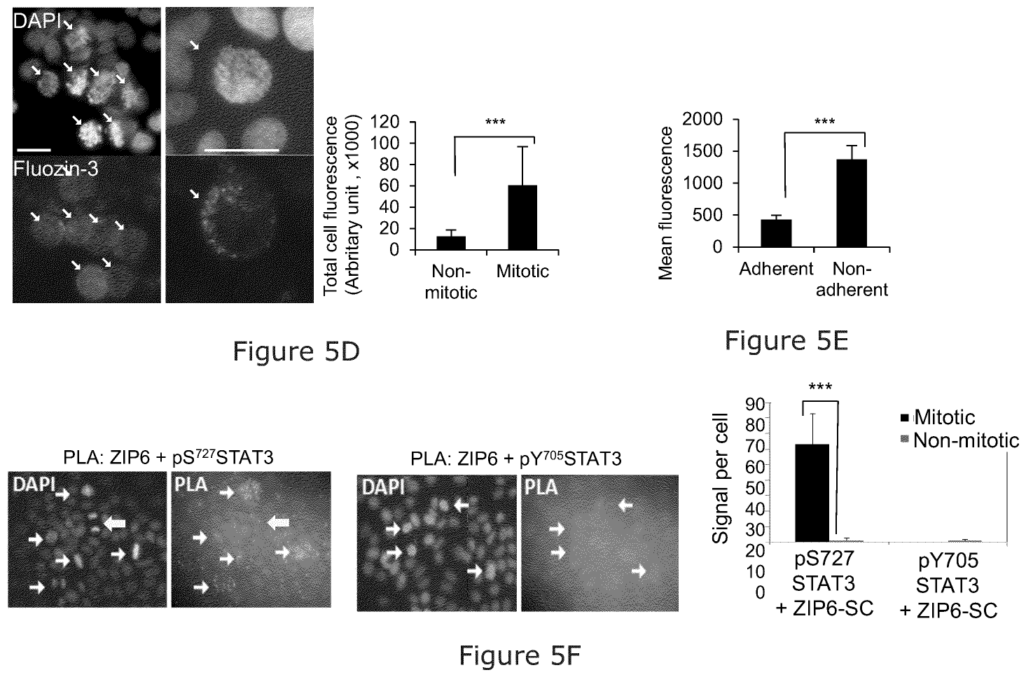

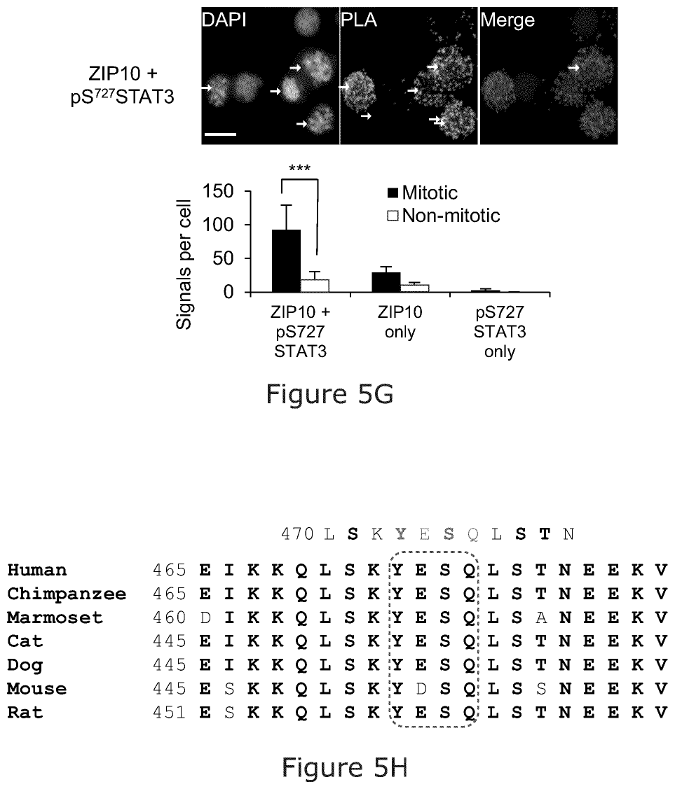

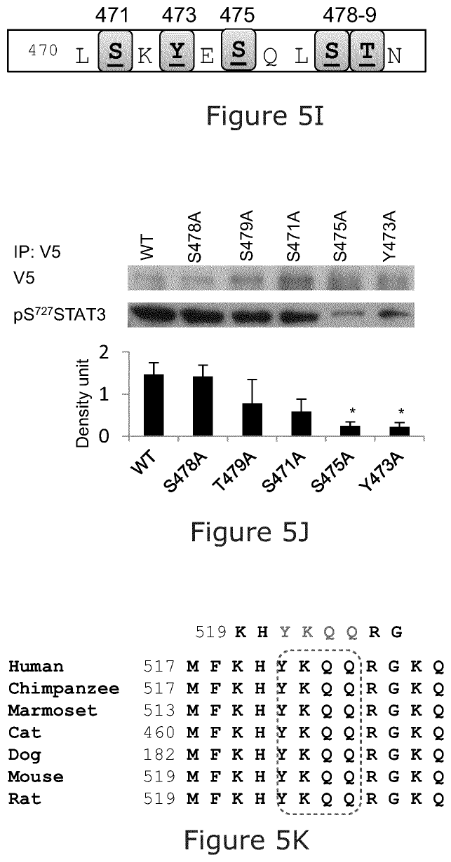

FIG. 2. Shows the requirement for ZIP6:ZIP10 heterodimer for mitosis. [A] FACS cell cycle analysis confirms the increase in the G2/M population in nocodazole-treated cells, compared to control. Results of three independent experiments are demonstrated as mean±SD. Statistical significance is determined by Student's t test. *** p<0.001. [B] Control PLA using either our ZIP6-Y or our ZIP10 antibodies alone in nocodazole-treated MCF-7 cells produces only a few dots in mitotic cells (arrow), compared to FIG. 3D. Scale bar, 20 μm. [C] Immunoprecipitation with V5 antibody in cells transfected with ZIP6 wild-type (WT) and probing for ZIP10 demonstrate the binding of recombinant ZIP6 and ZIP10, which is absent in the control IgG lane. [D] FACS cell cycle analysis confirms the increase in the G2/M population in the non-adherent cells collected using the mitotic shake-off technique, compared to the adherent cells from the same dishes. Results of three independent experiments are demonstrated as mean±SD. Statistical significance is determined by Student's t test. *** p<0.001. [E] PLA using ZIP6-SC and pY⁷⁰⁵STAT3 antibodies together as well as individual control ZIP6-SC and pS⁷²⁷STAT3 antibodies in nocodazole-treated MCF-7 cells produces only few dots, compared to FIG. 5F. Results of three independent experiments are demonstrated as mean±SD.

FIG. 3. The requirement of ZIP6:ZIP10 heterodimer for mitosis. [A] ZIP10 antibody treatment with 100 nM nocodazole for 20 hours significantly decreases the number of mitotic cells (positive for pS10HistoneH3, red) in a concentration-dependent manner. Scale bar, 25 μm. [B] FACS cell cycle analysis reveals a significant decrease in the G2/M population in MCF-7 cells treated with the ZIP10 antibody (1:20). [C-D] ZIP10 antibody treatment in MDA-436 (C) and MDA-231 cells (D), with 100 nM nocodazole for 20 hours significantly decreases the number of mitotic cells (positive for pS¹⁰HistoneH3, red) in a concentration-dependent manner. [E] Proximity ligation assay (PLA) using ZIP6-Y and ZIP10 antibodies in nocodazole-treated MCF-7 cells, produces increased dots in mitotic cells (white arrows) compared to non-mitotic cells. Scale bar, 15 μm. [F] Cell growth is significantly suppressed by treatment with either our ZIP6-Y or ZIP10 antibody. Results of at least three independent experiments are demonstrated as mean±SD. For A, C and D, statistical significance is compared to nocodazole-treated samples. For F, statistical significance is compared to control (Con). * p<0.05, ** p<0.01, *** p<0.001.

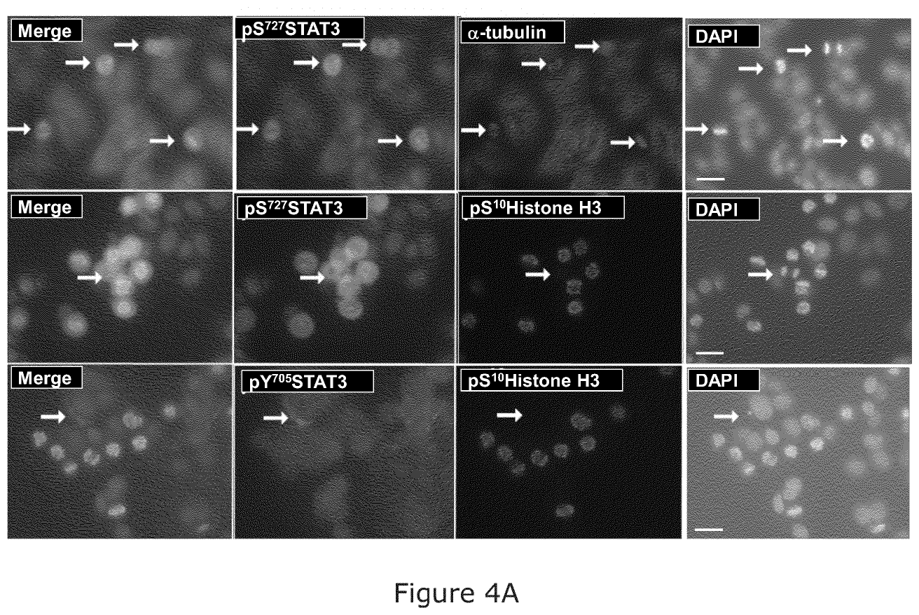

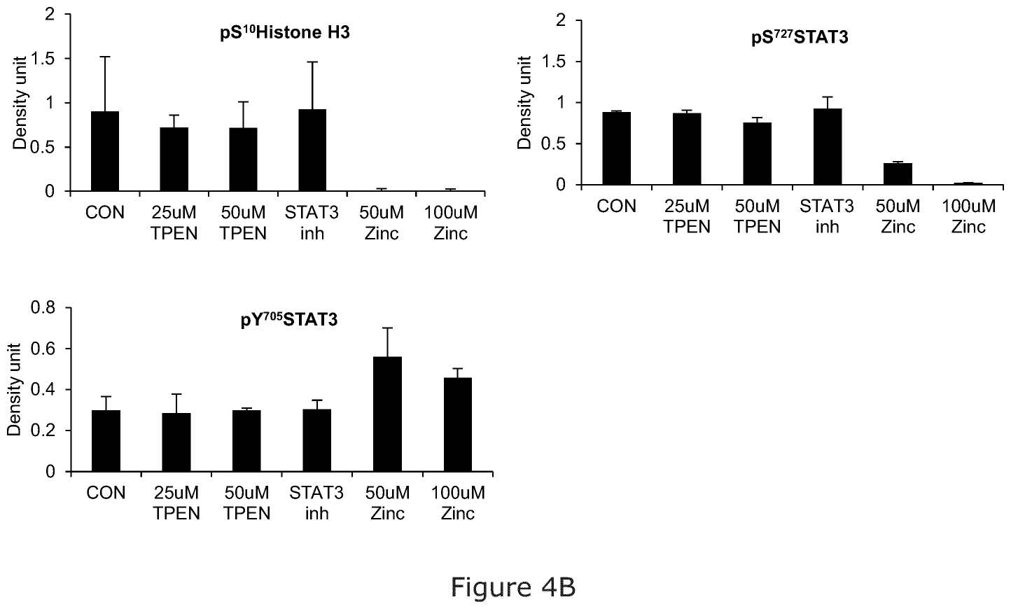

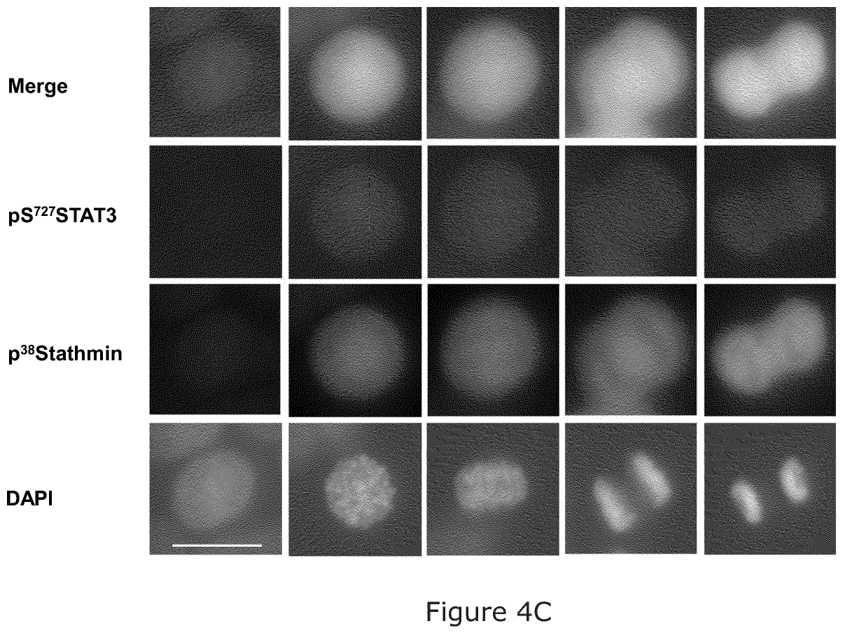

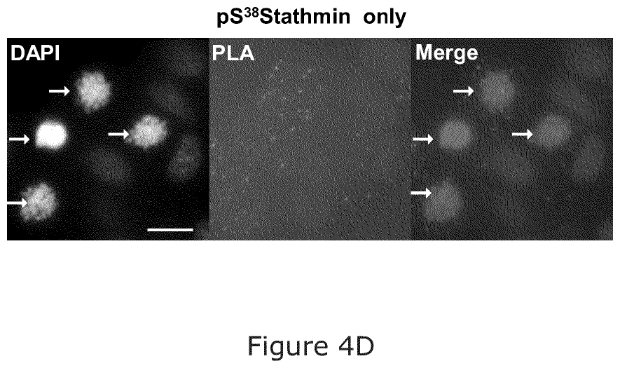

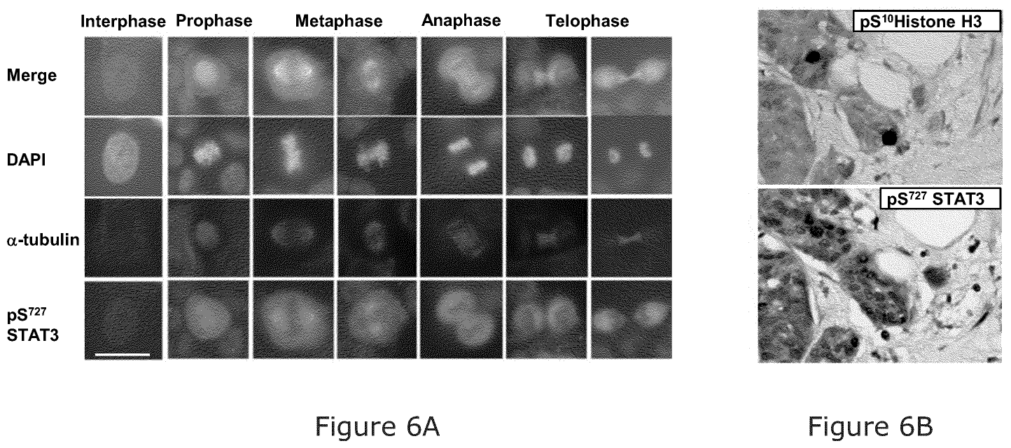

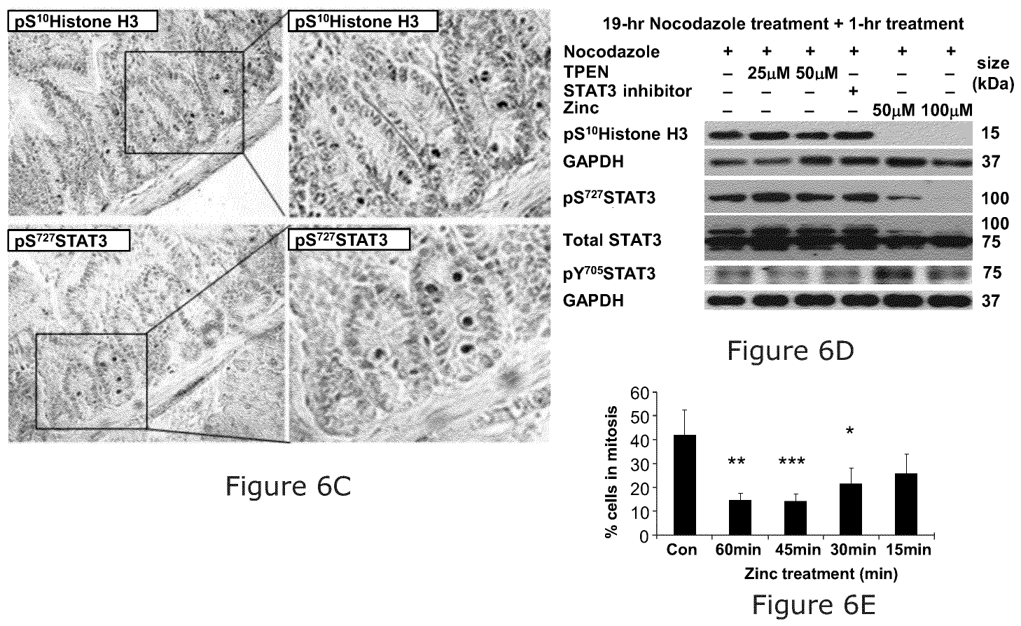

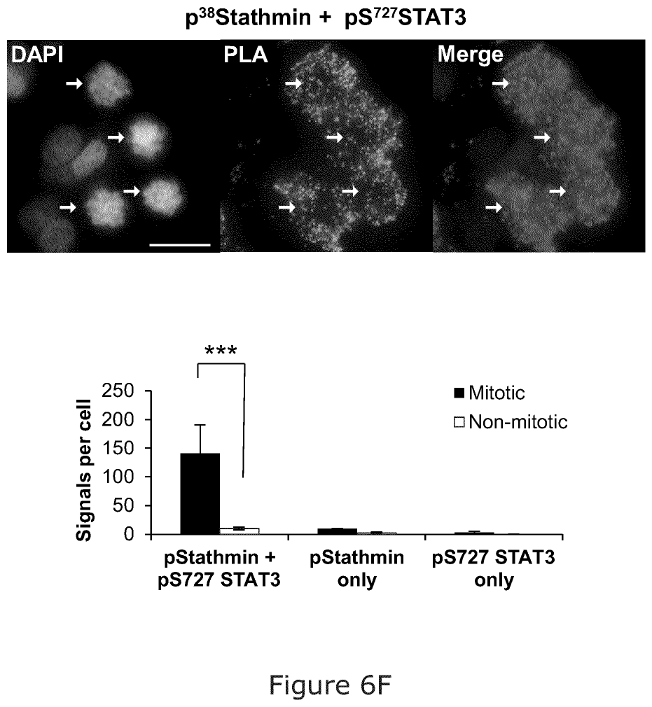

FIG. 4. The involvement of STAT3 in mitosis. [A] TAMR cells were immunostained for pS⁷²⁷STAT3 (green), pY⁷⁰⁵STAT3 (green), α-tubulin (red) and pS¹⁰HistoneH3 (red), and counterstained with DAPI (blue). Top panel: Mitotic cells (arrows) are positive for pS⁷²⁷STAT3. Middle panel: Arrow shows a cell in cytokinesis that is negative for pS10HistoneH3, yet still positive for pS⁷²⁷STAT3. Bottom Panel: Mitotic cells are negative for pY⁷⁰⁵STAT3. Arrow indicates pY⁷⁰⁵STAT3 staining on the plasma membrane. Scale bar, 20 μm. [B] Densitometric data for pS⁷²⁷STAT3, pY⁷⁰⁵STAT3 and pS¹⁰HistoneH3 normalised to GAPDH values from FIG. 6D are demonstrated as mean±SD. [C] Fluorescence microscopy shows co-localisation of pS³⁸Stathmin and pS⁷²⁷STAT3 during mitotic cell cycle phases, but not interphase. Scale bar, 10 μm. [D] Control PLA using pS³⁸Stathmin antibody alone produces only few dots in mitotic cells (arrows), compared to FIG. 6F. Scale bar, 15 μm.

FIG. 5. ZIP6 and ZIP10 bind zinc-triggered pS⁷²⁷STAT3 in mitosis. [A] Mitotic MCF-7 cells, stained for ZIP6-SC (red), pS⁷²⁷STAT3 (green) and DAPI (blue), and above the plain of adherent cells, are enriched for ZIP6 and pS⁷²⁷STAT3. Scale bar, 20 μm. [B] Increased pS⁷²⁷STAT3 and ZIP6 (68 kDa band) using ZIP6-SC antibody, and decreased pY⁷⁰⁵STAT3 in mitotic MCF-7 cells (pS¹⁰HistoneH3 staining) treated with 100 mM nocodazole. [C] MCF-7 cells treated with 20 μM zinc (Zn) and 10 μM pyrithione (P) in serum-free medium for 20 min shows a significant zinc-dependent increase in pS⁷²⁷STAT3 and a corresponding decrease in pY⁷⁰⁵STAT3. [D] MCF-7 cells loaded with 5 μM Fluozin-3 have increased green fluorescence only in mitotic cells (white arrow), total cell fluorescence confirms a statistically significant increase. Scale bar, 15 μm. [E] FACS analysis of nocodazole-treated MCF-7 cells, separated into adherent and non-adherent populations and loaded with 5 μM Fluozin-3, show increased green fluorescence. [F] PLA using ZIP6-SC and pS⁷²⁷STAT3 antibodies in nocodazole-treated MCF-7 cells produce a significantly increased number of dots only in mitotic cells (white arrows), in contrast to ZIP6-SC and pY⁷⁰⁵STAT3 antibodies. Yellow arrow indicates fewer dots in cytokinesis. [G] PLA using ZIP10 and pS⁷²⁷STAT3 antibodies in nocodazole-treated MCF-7 cells produces increased dots (white arrows) in mitotic cells. Scale bar, 15 μm. [H] Schematic of ZIP6 sequence in different species shows conservation of the predicted STAT3-binding site YESQ (Green box). [I] Schematic of mutated residues around STAT3-binding site in ZIP6. J. Immunoprecipitation with V5 antibody in cells transfected with ZIP6 wild-type (WT) or sequence-verified mutants, all with a C-terminal V5 tag, demonstrates a significant decrease in binding to pS⁷²⁷STAT3 in S475A and Y473A mutants. K. Schematic of ZIP10 sequence in different species demonstrates conservation of the predicted STAT3-binding site YKQQ (Green box). Results of at least three independent experiments are demonstrated as mean±SD. * p<0.05, *** p<0.001.

FIG. 6. The increase of pS⁷²⁷STAT3 throughout mitosis and its binding to pStathmin. [A] pS⁷²⁷STAT3 (green) is increased in all stages of mitosis in TAMR cells stained with DAPI (blue) and α-tubulin (red) compared to interphase. Scale bar, 12 μm. [B] Adjacent slices of breast cancer tissue show both pS¹⁰HistoneH3 and pS⁷²⁷STAT3 in the same mitotic cells. [C] Both pS¹⁰HistoneH3 and pS⁷²⁷STAT3 are present in mitotic cells within the crypts of normal mouse intestine. [D] Reduction of zinc (zinc chelator TPEN) or STAT3 inhibitor for 1 hour in TAM R cells pre-treated with nocodazole for 19 hours has no effect whereas zinc-treated samples loose pS¹⁰HistoneH3 and pS⁷²⁷STAT3 and full-length STAT3 band. [E] Mitotic count (positive for pS¹⁰HistoneH3) was performed in TAMR cells treated with 100 nM nocodazole for 19 hours plus 100 μM zinc and 10 μM pyrithione for 0-60 mins. 15 min zinc treatment is sufficient to progress through mitosis. [F] PLA using pS³⁸Stathmin and pS⁷²⁷STAT3 antibodies in nocodazole-treated MCF-7 cells produces increased dots in mitotic cells (white arrows) only. Scale bar, 20 μm. Results of at least three independent experiments are demonstrated as mean±SD. * p<0.05, ** p<0.01, *** p<0.001.

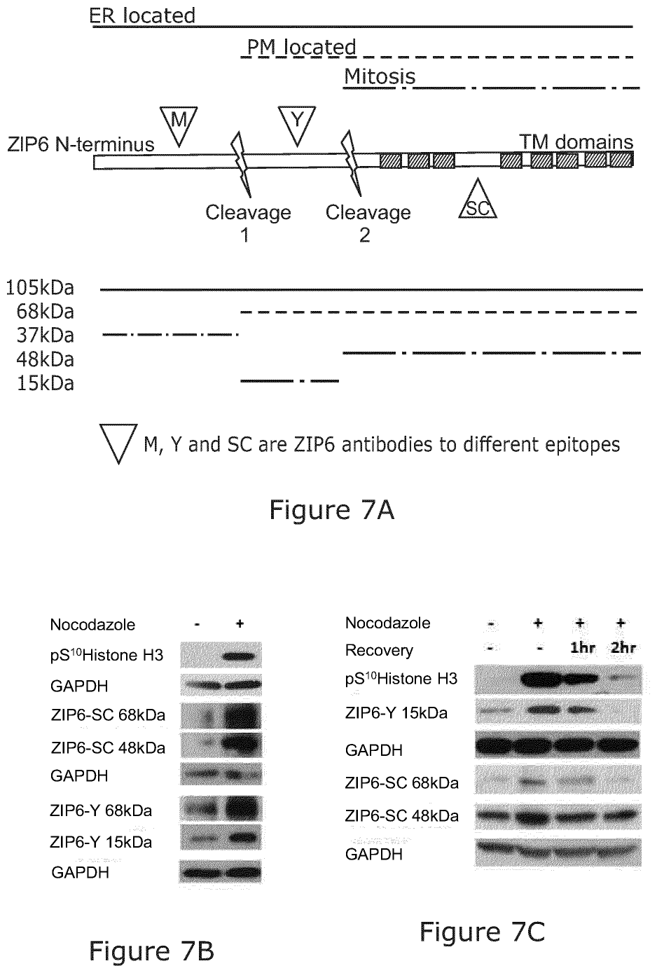

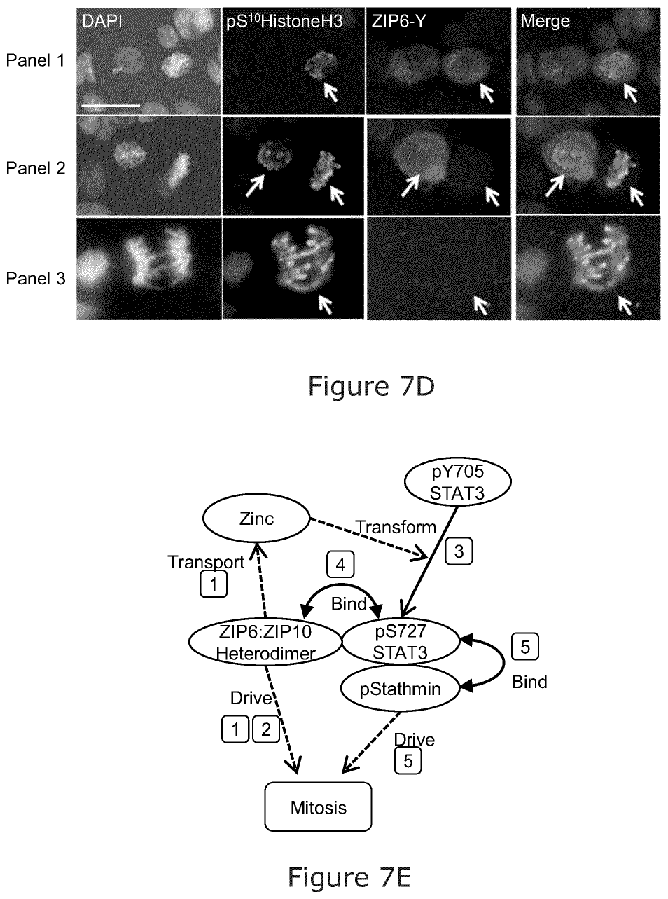

FIG. 7. Shows the N-terminal cleavage of ZIP6 during mitosis. [A] Schematic shows ZIP6 antibody epitope locations (M, Y and SC) and the band sizes obtained after cleavage 1 (PM relocation) and cleavage 2 (mitosis). [B] Nocodazole-treated adherent and non-adherent cells have increased pS¹⁰HistoneH3 and increased ZIP6 bands 68 kDa (SC and Y antibody), 48 kDa (SC antibody) and 15 kDa (Y antibody). [C] MCF-7 cells treated with 100 nM nocodazole for 20 hours with 1-2 hour recovery shows mitotic progression (positive for pS¹⁰HistoneH3) and mirrored by the ZIP6 bands, including 15 kDa (ZIP6-Y antibody), consistent with cleavage 2. [D] MCF-7 cells stained for pS¹⁰HistoneH3 (green, arrows), ZIP6-Y (red) and DAPI (blue) demonstrate the presence of ZIP6-Y in prophase (Panel 1-2) and absence in both metaphase (Panel 2) and anaphase (Panel 3). Scale bar, 20 μm. [E] Schematic demonstrating a model of ZIP6:ZIP10 heterodimer importing zinc into cells and driving mitosis (1, 2). The imported zinc triggers formation of pS⁷²⁷STAT3 (3), which binds ZIP6 and ZIP10 (4) and pS³⁸Stathmin (5), linking pS⁷²⁷STAT3 to the pS³⁸Stathmin-driven microtubule re-organisation needed for mitosis;

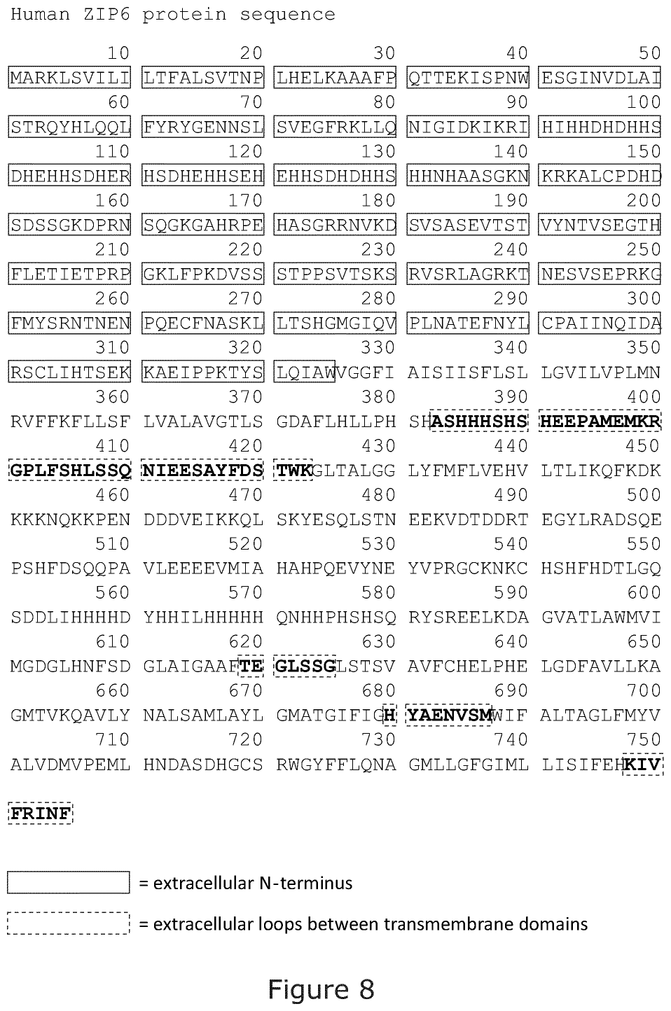

FIG. 8. ZIP6 protein sequence (SEQ ID NO: 19) showing N-terminal region and extra-cellular loops for binding according to the invention;

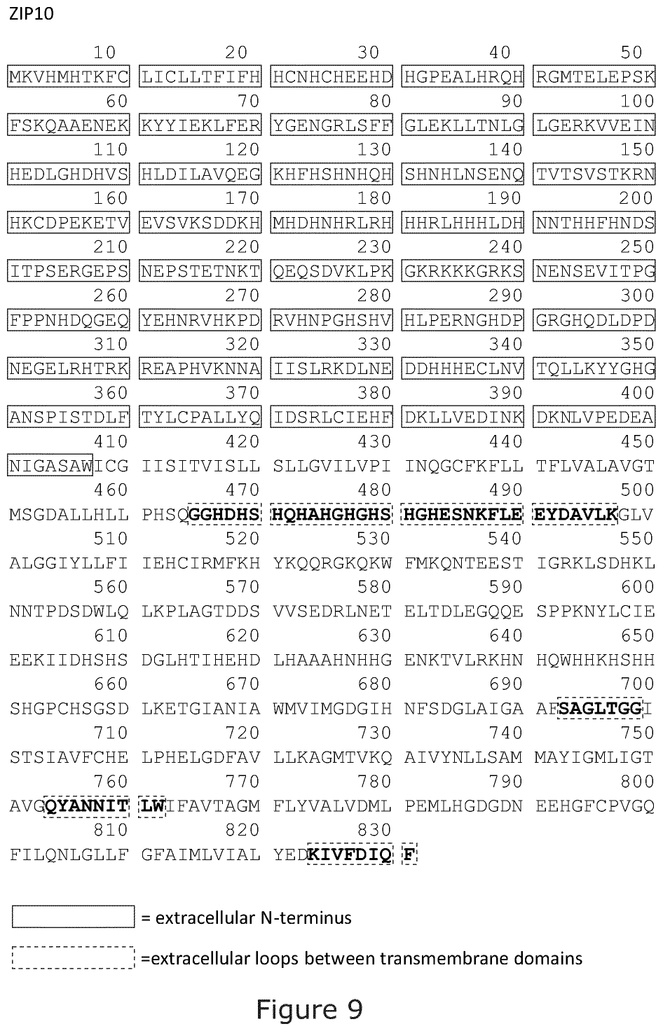

FIG. 9. ZIP10 protein sequence (SEQ ID NO: 20) showing N-terminal region and extra-cellular loops for binding according to the invention;

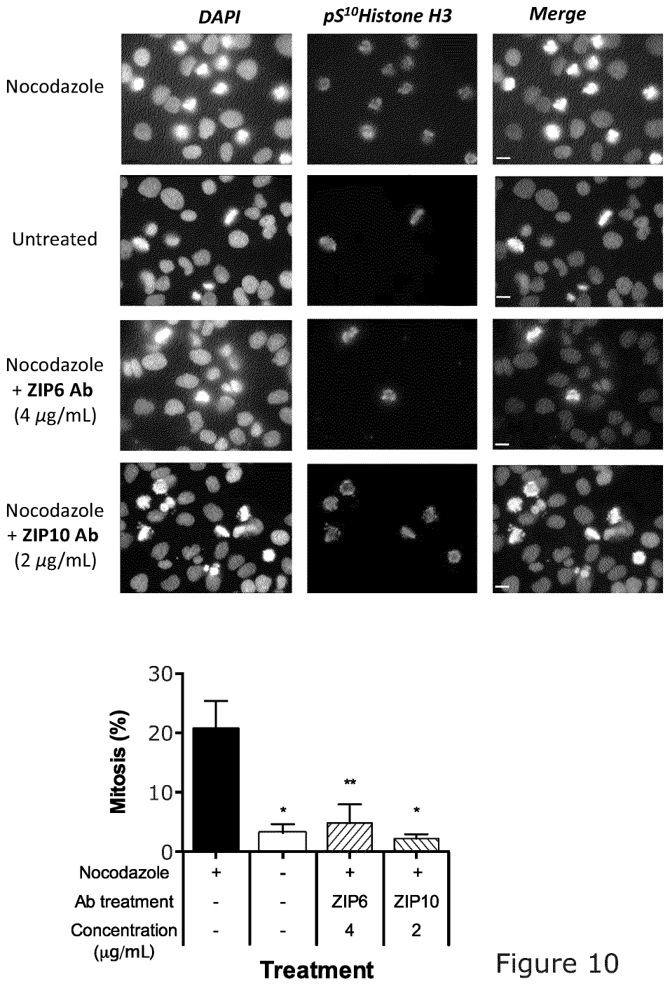

FIG. 10. ZIP6 and ZIP10 antibody inhibition of mitosis in melanoma cells. SK-MEL-29 cells were treated with 150 nM nocodazole for 18 hours with or without ZIP6Y or ZIP10 antibodies. The cells were stained for pS¹⁰Histone H3 (red) and DAPI (blue). A representative image of each population is shown. Scale bars: 10 μm. Graph shows percentages of cells in mitosis presented as mean±standard error (n=3). Statistical significance was measured using ANoVA, comparing to the population treated with nocodazole alone. * p<0.05, ** p<0.01;

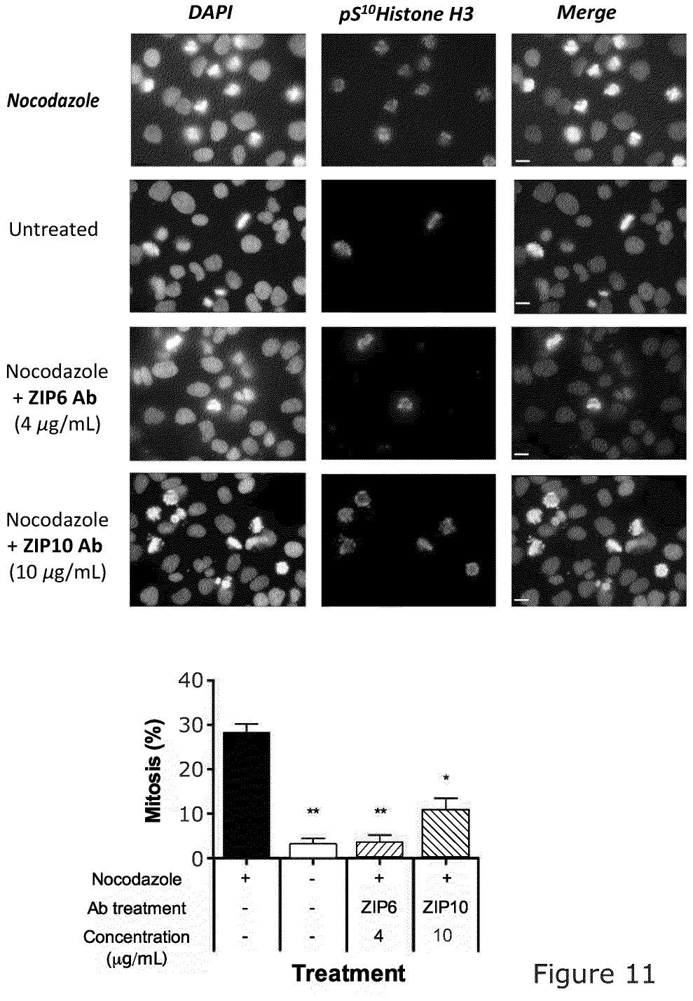

FIG. 11. ZIP6 and ZIP10 antibody inhibition of mitosis in prostate cancer cells. DU145 cells were treated with 150 nM nocodazole for 18 hours with or without ZIP6Y or ZIP10 antibodies. The cells were stained for pS¹⁰Histone H3 (red) and DAPI (blue). A representative image of each population is shown. Scale bars: 20 μm. Graphs shows percentages of cells in mitosis presented as mean±standard error (n=3). Statistical significance was measured using ANoVA, comparing to the population treated with nocodazole alone. * p<0.05, ** p<0.01;

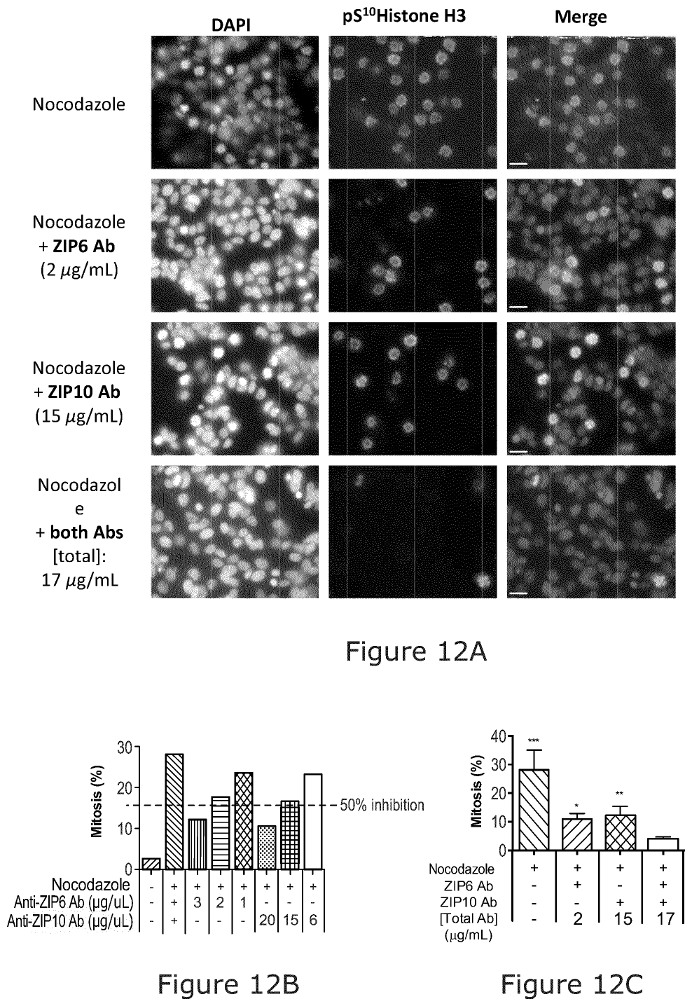

FIG. 12. ZIP6 and ZIP10 combined antibody inhibition of mitosis in MCF-7 cells using both ZIP6Y and ZIP10 antibodies. MCF7 cells were treated with 150 nM nocodazole for 18 hours with or without the ZIP6 or ZIP10 antibodies or a mixture of the two (A) the amount of each antibody used was judged from the observed percentage of cells in mitosis from a dose giving a 50% reduction of the cells in mitosis (pink line) in B. The cells were stained for pS¹⁰Histone H3 (red) and DAPI (blue). A representative image of each population is shown. Square brackets denote total concentration of the two antibodies together. Scale bars: 10 μm. C. Percentages of cells in mitosis are presented as mean±standard error (n=3). Statistical significance was measured using ANoVA, comparing to the population treated with both antibodies together. * p<0.05, ** p<0.01, *** p<0.001;

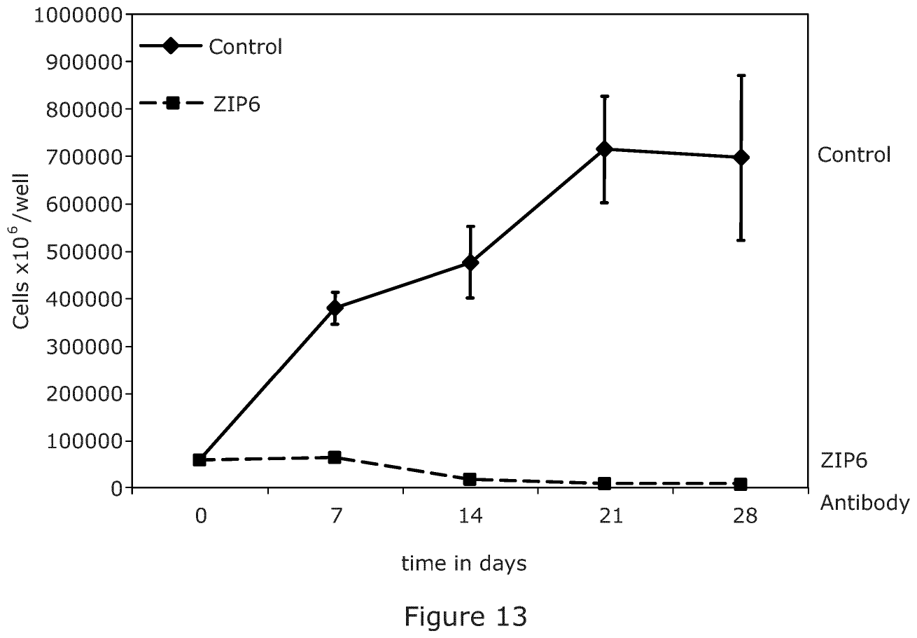

FIG. 13. ZIP6 antibody inhibition of cell division in MCF-7 cells over 28 days. MCF-7 cells were treated with ZIP6Y antibody when the medium was changed twice per week for 28 days in total. The total cell number was counted using a coulter counter and cells were harvested weekly. Graph represents mean±standard error (n=3);

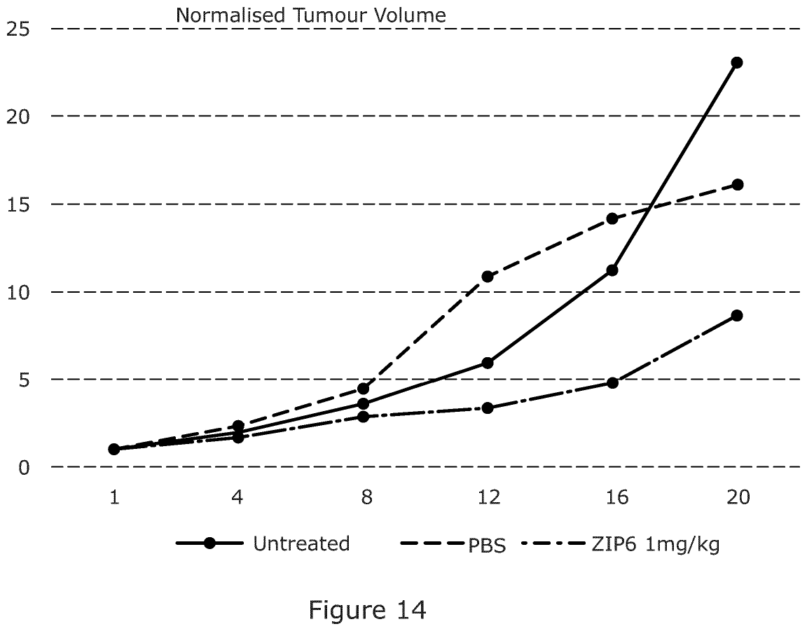

FIG. 14. ZIP6Y antibody injections slow triple negative breast cancer tumours in vivo by over 50%. Mice, injected with MDA 231 triple negative breast cancer cells, developed tumours after which mice were injected with antibody or controls every 4 days for 20 days and tumour volume was measured. ZIP6 treated tumours grew 50% less compared to PBS controls and 66% less compared to untreated in 20 days.

Table 1. List of N-terminal ZIP6 and ZIP10 antibodies.

Materials and Methods

Materials, Antibodies and Treatments

Antibodies used were ZIP6-Y and ZIP10 (in house); ZIP6-SC (E-20, SC-84875), pS⁷²⁷STAT3 (SC-8001-R and SC-136193), pY⁷⁰⁵STAT3 (SC-7993-R), total STAT3 (SC-8019) and GAPDH (SC-32233) from Santa Cruz Biotechnology; α-tubulin (DM1A, #3873S), pS¹⁰HistoneH3 (#9706S and #3377) and pS³⁸Stathmin (#4191) from Cell Signalling Technology; mouse V5 from Invitrogen; rabbit V5 (Ab9116) from Abcam; β-actin (A5316) from Sigma-Aldrich.

Treatments used were 100 ng/mL nocodazole (Sigma-Aldrich, M1404) 20 hours, 200 μM STAT3 inhibitor cell-permeable peptide (Calbiochem, 573096), and 20-100 μM zinc with 10 μM sodium pyrithione (Sigma-Aldrich), and 25 or 50 μM TPEN (Sigma-Aldrich).

Cell Lines and Immunohistochemistry

MCF-7 cells and tamoxifen-resistant cells (TAMR) developed from MCF-7 cells (1) were cultured as previously described (2). Formalin-fixed paraffin-embedded breast cancer samples were dewaxed and rehydrated before incubated with pS⁷²⁷STAT3 (SC-8001-R, 1/650) or pS¹⁰HistoneH3 (#3377, 1/30) antibodies for 2 hours and detected with Dako Envision #K4011 reagent. Two-minute pressure cooker at pH9 in Tris base plus EDTA was used for antigen retrieval. The primary breast cancer material used had correct ethical approval (REC reference number C2020313). Immunohistochemistry of mouse intestinal tissue was previously described (3).

Plasmids and Transfections

The generation of recombinant constructs for ZIP6/LIV-1/SLC39A6 (4) and ZIP7/HKE4 (5) with C-terminal V5 tags using vector pcDNA3.1/V5-His-TOPO has been previously described. ZIP6 mutants (Y473A, S471A, S475A and 5478A, S479A) were generated from the above plasmid and confirmed by sequencing. Cells were transfected with Lipofectamine-2000 (Life Technologies) for 16 hours as described (6).

SDS-PAGE, Western Blotting and Immunoprecipitation.

Cells were harvested, washed with PBS, lysed for 1 hour at 4° C. with lysis buffer pH7.6 (50 mM Tris, 150 mM NaCl, 5 mM EGTA and 1% Triton X-100) with protease inhibitor cocktail for mammalian cells (Signa-Aldrich) and phosphatase inhibitors (2 mM sodium orthovanadate and 50 mM sodium fluoride). Protein was measured using Bio-Rad/Bradford dye-binding protein microassay. Western Blot results of 40 μg/lane from three separate experiments were normalised to GAPDH values. For immunoprecipitations, 500 μg of protein was incubated with 5 μg of antibody overnight and 20 μl of EZview Red Protein A Affinity Gel (Sigma) for 4 hours prior to washing and SDS-Page.

Fluorescence Microscopy and FACS Analysis

1×10⁵ cells were grown on 0.17 mm thick coverslips for 5-7 days prior to transfection. Coverslips were fixed and processed as previously described (7). For zinc imaging, cells were loaded with 5 μM Fluozin-3 (Invitrogen) for 30 min at 37° C. For FACS analysis using a Becton-Dickinson FACSVerse, non-adherent cells collected by mitotic shake-off and adherent cells harvested by trypsinisation were loaded with 5 μM Fluozin-3 (Invitrogen) for 30 min followed by 30 min recovery in medium, For cell cycle analysis, cells were fixed in 70% ethanol overnight followed by DNA staining with 20 μg/mL propidium iodide (Sigma-Aldrich) plus 0.2 μg/mL DNase-free RNase A and 0.1% Triton X-100 in PBS at 37° C. for 20 min before FACS analysis and analysed with FlowJo Software version 10 using Watson pragmatic algorithm. Scale bar is 10 μm.

Proximity Ligation Assay (PLA)

Cells on eight-well chamber slides (Lab-Tek, Fisher) were fixed followed by PLA using Duolink red kit (Sigma) as described previously (8) and where red fluorescent dots indicate two molecules binding. Dots per cell were determined with ImageTool software (Olink) using at least 12 separate images from at least three different experiments and presented as average values±standard errors.

Statistical Analysis

Statistical analysis was performed using either Student's t-test or ANoVA with Post-Hoc Dunnett and Tamhane tests. Significance was assumed with *=p<0.05, **=p<0.01, ***=p<0.001. Error bars are standard deviation (SD) with at least 3 different experiments.

Results

The Requirement of ZIP6 and ZIP10 for Mitosis.

We first discovered a role for ZIP6 in mitotic initiation by incubating cells with our ZIP6-Y N-terminal antibody. Cells treated with nocodazole, which blocks microtubule polymerisation, have an increased percentage of mitotic cells, as judged by pS¹⁰HistoneH3 and DAPI staining (FIG. 1A) and a significant increase of G2/M cells by FACS analysis (FIG. 2). Nocodazole with our ZIP6-Y antibody treatment significantly reduced mitosis in a concentration-dependant manner (FIG. 1A) and decreased the G2/M tetraploid population (FIG. 1B), suggesting blockade of ZIP6-mediated zinc influx.

This effect was reproduced in two triple negative (ER−, PR− and Her2−) breast cancer cell lines (MDA-436 and MDA-231; FIGS. 1C and 1D), and further in melanoma cancer cell lines (SK-MEL-29; FIG. 10) and prostate cancer cell lines (DU145; FIG. 11). Notably, this effect was also replicated in vivo, with it demonstrated that ZIP6 inhibition slowed growth of difficult to treat triple negative breast cancer (TN BC) tumours by over 50% compared to controls (FIG. 13).

To confirm that blocking ZIP6 did not work by stabilising the microtubules and thus helping the cells to overcome nocodazole block, we repeated this experiment without nocodazole by adding ZIP6-Y antibody before cells entered mitosis, at 30 hours after partial synchronisation by 24-hour serum withdrawal, which significantly decreased mitosis (FIG. 1E). Furthermore, cells transfected with ZIP6, in contrast to LacZ or ZIP7, increased the mitotic percentage by two-fold in adherent cells and fourfold in non-adherent cells (FIG. 1F), further confirming the important role of ZIP6 in mitosis.

Since we have shown ZIP6 and ZIP10 form a heterodimer, we tested whether ZIP10 behaved similarly to ZIP6 in mitosis. Incubation of nocodazole-treated cells with our ZIP10 antibody similarly decreased the mitotic population (FIG. 2A), confirmed by FACS analysis (FIG. 2B) and also observed in MDA-436 cells (FIG. 2C), MDA-231 cells (FIG. 2D), SK-MEL-29 cells (FIG. 10) and DU145 cells (FIG. 11)

Given that ZIP6 or ZIP10 inhibition equally prevented mitosis we tested ZIP6 and ZIP10 binding using proximity ligation assay, which revealed binding enrichment in mitotic cells (FIG. 3E, 4A), an observation confirmed by immunoprecipitation with V5 in ZIP6-transfected cells which were subsequently probed for ZIP10 (FIG. 4B). Additionally, treatments with ZIP6-Y or ZIP10 antibody significantly suppressed cell growth over 96 hours (FIG. 3F), demonstrating that blocking these zinc transporters prevents cell division, which was shown to be the case even up to 28 days (FIG. 13).

Further still, we also explored the effect of combined inhibition of the ZIP6/ZIP10 heterodimer through treatment with both a blocking ZIP6 and ZIP10 used in combination (FIG. 12). Notably, combined treatment led to an even further reduction in mitosis, compared to either antibody alone, suggesting combined treatment may offer superior inhibition of tumour proliferation (FIG. 12C).

ZIP6 and ZIP10 Bind Zinc-Triggered pS⁷²⁷STAT3 in Mitosis

ZIP6 was enriched in rounded mitotic cells (FIG. 5A), and we discovered these mitotic cells also contained pS⁷²⁷STAT3. We confirmed the elevated pS⁷²⁷STAT3 in mitosis (FIG. 5B), by Western blot with a concurrent reduction of pY⁷⁰⁵STAT3 (FIG. 5B) and an increased 68 kDa ZIP6 band, consistent with N-terminal cleavage and plasma membrane location. Furthermore, zinc treatment changes the phosphorylation status of STAT3 from pY705 to pS727 (FIG. 5C), demonstrating a reciprocal relationship, which has been previously reported yet not in a mitotic context. However, this detection of pS⁷²⁷STAT3 in mitosis was also observed in a mitotic whole genome mass spectrometry screen. Interestingly, loss of pY⁷⁰⁵STAT3 will terminate the transcriptional activity of STAT3 and transcription is known to cease during mitosis.

We next confirmed an increase in zinc in mitotic cells (FIG. 5D, E), showing a 3-fold increase in non-adherent cells. These non-adherent cells were enriched with mitotic cells, as 90% had 4N DNA content (FIG. 4A) compared to 48% for adherent cells.

Others have also observed increased zinc in mitotic cells demonstrating a threefold zinc increase in mitotic versus interphase cells.

Using proximity ligation assay we next demonstrated significant binding exclusively in mitotic cells of both ZIP6 (FIG. 5F) and ZIP10 (FIG. 5G) separately to pS⁷²⁷STAT3. In contrast there was no detectable interaction between either ZIP6 or ZIP10 with pY⁷⁰⁵STAT3 (FIG. 4B). Interestingly, less ZIP6 was bound to pS⁷²⁷STAT3 after cytokinesis (FIG. 5F, yellow arrow) suggesting dissociation when mitosis ceased. These data demonstrate the zinc-mediated formation of pS⁷²⁷STAT3 which binds ZIP6 and ZIP10 exclusively during mitosis.

The ZIP6 protein sequence has a predicted STAT3-binding site on the cytoplasmic loop (YESQ, residues 473-476) between transmembrane domains 3-4, which fits the consensus motif of a STAT3-binding site YxxQ and is highly conserved in mammals (FIG. 5H). We generated ZIP6 mutants (FIG. 5I) to investigate whether any residues were involved in binding STAT3 and tested this by immunoprecipitation. Probing for pS⁷²⁷STAT3 co-precipitated ZIP6 in cells transfected with both wild-type ZIP6 and mutants S471A, S478A and T479A but not with Y473A or S475A mutants (FIG. 5J). These data are consistent with STAT3 binding ZIP6 at residue Y473 and S475 is also important. These residues reside in an evolutionary conserved sequence of ZIP6 (FIG. 5H). ZIP10 also has a well conserved predicted STAT3-binding site YKQQ (512-524) in the corresponding region (FIG. 5K).

pS⁷²⁷STAT3 Binds pS³⁸Stathmin During Mitosis and is Cleaved for Mitosis Exit

To dissect the temporal association of pS⁷²⁷STAT3 with the mitotic process, we imaged pS⁷²⁷STAT3 in cells during mitosis (FIG. 6A). This revealed the presence of pS⁷²⁷STAT3 at all mitotic stages which was confirmed by pS⁷²⁷STAT3 presence and pY⁷⁰⁵STAT3 absence in mitotic cells, noting that cells undergoing cytokinesis were still positive for pS⁷²⁷STAT3 yet negative for pS¹⁰HistoneH3, indicating the prolonged presence of pS⁷²⁷STAT3 throughout mitosis. Furthermore, we detected pS⁷²⁷STAT3 in mitotic cells in vivo, as judged by pS¹⁰HistoneH3 in the same cells using adjacent slices of human breast cancer (FIG. 6B). Additional in vivo staining of pS⁷²⁷STAT3 was also seen in the mitotic cells of normal mouse intestine (FIG. 6C), suggesting a common process during mitosis that encompasses normal and disease states.

The effect of zinc on mitotic progression was examined by treatment with either a zinc chelator or STAT3 inhibitor for the last hour of nocodazole treatment and had no effect on pS¹⁰HistoneH3 or STAT3 phosphorylation status (FIG. 6D), establishing that both STAT3 and zinc were required before cells reached mitosis. Interestingly, 1-hour incubation with 50 μM or 100 μM zinc had effects consistent with cells no longer in mitosis, as judged by loss of pS¹⁰HistoneH3 and also the reversion of pS⁷²⁷STAT3 to pY⁷⁰⁵STAT3 (FIG. 6D). We confirmed that these cells had progressed through mitosis by fluorescent imaging using a reduced zinc exposure time (FIG. 6E) and observed loss of mitotic cell number as soon as 15 min. Importantly, these zinc-treated cells which had exited mitosis had lost the usual full length STAT3 band (FIG. 6D), showing only the C-terminally cleaved form of STAT3, thus removing residue S727. This cleavage of STAT3 at the end of mitosis, removing S727, will enable the Y705-phosphorylated active transcription factor form of STAT3 to be re-instated, so re-establishing transcription capability and providing a clear means to exit mitosis.

We integrated the ZIP6:pS⁷²⁷STAT3 complex into the established mitotic cascade by demonstrating an interaction of pS⁷²⁷STAT3 with pS³⁸Stathmin. This form of Stathmin not only enables mitotic microtubule reorganisation but it is also essential for both mitotic spindle assembly and mitotic entry. Therefore, establishing the presence and binding of pS⁷²⁷STAT3 with pS³⁸Stathmin exclusively in mitotic cells (FIG. 6F) supports a role for pS⁷²⁷STAT3 in stabilising Stathmin during mitosis.

The N-terminal cleavage of ZIP6 during mitosis We have previously established N-terminal cleavage of ZIP6 as a requirement for re-location to the plasma membrane at a predicted PEST site [7], and since these are often processed in a cell cycle-regulated manner we examined any additional cleavage during mitosis using ZIP6 antibodies with different epitopes (FIG. 7A).

Nocodazole-treated cells showed a substantial increase in a 68-kDa band of ZIP6 (FIG. 7B), recognised by both the N-terminal ZIP6-Y antibody and the cytoplasmic loop ZIP6-SC antibody (FIG. 7A, cleavage 1). We also detected two shorter bands for ZIP6, both increased in nocodazole-treatment, corresponding to a 15-kDa band with ZIP6-Y antibody and a 48-kDa band with ZIP6-SC antibody (FIG. 7B), signifying further N-terminal cleavage (FIG. 7A, cleavage 2) and ecto-domain shedding of the remaining ZIP6 extracellular N-terminus. The prion protein, descended from the LIV-1 subfamily of ZIP channels, which includes both ZIP6 and ZIP10, has a similar N-terminus to the ZIP channels and undergoes ecto-domain shedding as a means of functional control. Interestingly, N-terminal cleavage is required for proper zinc-transport function of both ZIP10 and ZIP4, two members of the LIV-1 family of zinc transporters. These facts together with our data provide new evidence that extracellular cleavage of N-terminal ZIP6 and/or ZIP10 and may be required to regulate the ZIP6:ZIP10 heterodimer function and the ability to trigger the onset of mitosis.

To examine the exact relationship of ZIP6 cleavage 2 with mitotic progression, we investigated ZIP6 cleavage during 1-2 hours recovery after nocodazole treatment, allowing progression through mitosis as verified with decreasing pS¹⁰HistoneH3 (FIG. 7C). In these conditions, we observed an increase in the ZIP6 15-kDa band in parallel with the activation of pS¹⁰HistoneH3, consistent with its appearance in mitosis (FIG. 7C), followed by a decrease as the cells moved out of mitosis. Furthermore, the ZIP6-SC antibody 68 kDa band was reduced and a band of 48-kDa appeared (FIG. 7C), consistent with the occurrence of ZIP6 cleavage 2 (FIG. 7A). Additionally, when imaging mitotic cells for ZIP6 by immunofluorescence, we were only able to observe ZIP6-Y positive mitotic cells during prophase (FIG. 7D, panels 1 and 2) with cells at subsequent stages of mitosis always negative for ZIP6-Y (FIG. 7D, panels 2 and 3), verifying the removal of the ZIP6 N-terminus after the start of mitosis. These data together confirm that ZIP6 is additionally cleaved at the N-terminus, downstream of the ZIP6-Y antibody epitope (FIG. 7A), after mitosis has been initiated in prophase. It is also noteworthy that the second N-terminal cleavage of ZIP6 only occurs after the zinc-dependant alteration of the phosphorylation state of STAT3, suggesting that this second cleavage may inactivate the ZIP6:ZIP10 heterodimer.

All these data together allow us to formulate a new mechanism for the previously elusive role of zinc in mitosis. We propose a model (FIG. 7E) which includes the formation of a mitotic complex (ZIP6, ZIP10, pS⁷²⁷STAT3 and pS³⁸Stathmin) that feeds into known mitotic pathways such as Stathmin-dependant microtubule reorganisation and HistoneH3 activation. The ZIP6:ZIP10 heterodimer, activated by ZIP6 N-terminal cleavage, moves to the plasma membrane to import zinc into cells and initiate mitosis. This imported zinc triggers the formation of pS⁷²⁷STAT3 which remains throughout mitosis preventing STAT3 transcription activity. This pS⁷²⁷STAT3 also binds both ZIP6 and ZIP10 as well as pS38Stathmin, forming a complex which links pS⁷²⁷STAT3 to the p38Stathmin-driven microtubule re-organisation needed for mitosis.

### SUMMARY

We have shown a requirement of zinc influx through a ZIP6/ZIP10 heterodimer before the mitosis pathways can be activated and this explains why zinc has been essential for cell growth, and additionally, why zinc deficiency can be so detrimental. The ability to inhibit mitosis with ZIP6 or ZIP10 antibodies, specifically those binding the N-terminus of these transporters, now offers new therapeutic opportunities for inhibiting proliferative diseases such as cancer and fibrosis.

TABLE 1
ZIP6 and ZIP10 N-terminal antibodies

| | | | |
|---|---|---|---|
| | | Company | info |
| Name | species | details | |

ZIP6

| | | | |
|---|---|---|---|
| ZIP6M | Mouse | In House | N-terminus epitope (SEQ ID NO: 1 93- |
| | monoclonal | | HHDHDHHSDHEHHSD-107) |
| ZIP6Y | Mouse | In House | N-terminus epitope (SEQ ID NO: 2 246- |
| | monoclonal | | EPRKGFMYSRNTNE-259) |
| ZIP6AM | Mouse | In house | N-terminus epitope (SEQ ID NO: 2 246- |
| | monoclonal | | EPRKGFMYSRNTNE-259) |
| ZIP6X | Mouse | Genscript custom | N-terminus, |
| | polyclonal | made for us | (SEQ ID NO: 2 246-EPRKGFMYSRNTNE-259) |
| ZIP6R | Rabbit | Biogenes custom | N-terminus, |
| | polyclonal | made for us | (SEQ ID NO: 2 246-EPRKGFMYSRNTNE-259) |
| Anti- | Rabbit | Abcam ab61307 | SEQ ID NO: 3 RSCLIHTSEK KAEIPPKTYS |
| SLC39A6^{a} | polyclonal | | LQIAWVGGFI AISIISFLSL LGVILVPLMN (301- |
| | | | 350), N-terminus and including Transmembrane |
| | | | domain 1 |
| Anti- | Rabbit | AVIVA systems | SEQ ID NO: 3 RSCLIHTSEK KAEIPPKTYS |
| SLC39A6^{b} | polyclonal | biology | LQIAWVGGFI AISIISFLSL LGVILVPLMN (301- |
| | | ARP43931_T100 | 350), N-terminus and including Transmembrane |
| | | | domain 1 |
| Anti- | Rabbit | Abcam ab183101 | N-terminus, SEQ ID NO: 4 303-315, |
| SLC39A6^{c} | polyclonal | | CLIHTSEKKAEIP |
| Anti- | Rabbit | Abcam ab133378 | Unknown 18 residue N-terminal peptide |
| SLC39A6^{d} | polyclonal | | |
| LIV-1/ZIP6 | Rabbit | Novus | N-terminus, SEQ ID NO: 5 129-263, |
| antibody | polyclonal | NBP2-13337 | HSHHNHAASGKNKRKALCPDHDSDSSGKDPR |
| | | | NSQGKGAHRPEHASGRRNVKDSVSASEVTSTV |
| | | | YNTVSEGITIFLETIETPRPGKLEPKDVSSSTPPS |
| | | | VTSKSRVSRLAGRKTNESVSEPRKGEMYSRNT |
| | | | NENPQE |
| Anti- | Rabbit | Sigma | N-terminus, SEQ ID NO: 5 129-263, |
| SLC39A6^{e} | polyclonal | HPA042377 | HSHHNITAASGKNKRKALCPDFIDSDSSGKDPR |
| | | | NSQGKGAHRPEHASGRRNVKDSVSASEVTSTV |
| | | | YNTVSEGTHFLETIETPRPGKLFPKDVSSSTPPS |
| | | | VTSKSRVSRLAGRKTNESVSEPRKGFMYSRNT |
| | | | NENPQE |
| Anti- | Rabbit | Sigma AV43931 | N-teminius, SEQ ID NO: 6 288-337, |
| SLC39A6^{f} | polyclonal | | NYLCPAIINQIDARSCLIHTSEKKAEIPPKTYSLQ |
| | | | IAWVGGFIAISIISF |
| ZIP62-A | Rabbit | Alpha Diagnostic | N-terminus, extracellular, 15amino acids, unknown |
| | polyclonal | international | |

ZIP10

| | | | |
|---|---|---|---|
| ZIP10/ZIP10- | Rabbit | In house | N-terminus, SEQ ID NO: 11 46-59 |
| R | polyclonal | LEPSKFSKQAAENE | |
| Anti- | Rabbit | Antibody verify | N-terminus, |
| SLC39A10^{a} | polyclonal | AAS04440C | |
| Anti- | Rabbit | Sigma | N-terminus, SEQ ID NO: 12 164-253 |
| SLC39A10^{b} | polyclonal | HPA036513 | EKETVEVSVKSDDKHMHDHNHRLRHHHRLHH |
| | | | HLDHNTHHFHNDSITPSERGEPSNEPSIETNKT |
| | | | QEQSDVKLPKGKRKKKGRKSNENSEVITPGFP |
| SLC39A10^{a} | Rabbit | Abgent AP5464c | N-terminus, SEQ ID NO: 13 295-395 |
| antibody | polyclonal | | QDLDPDNEGELRHTRKREAPHVKNNAIISLR |
| Anti- | Rabbit | Sigma | N-terminus, SEQ ID NO: 14 36-85 |
| SLC39A10^{c} | polyclonal | SAB2102209 | LHRQHRGMTELEPSKFSKQAAENEKKYYIEKL |
| | | | FERYGENGRLSFFGLEKL |
| SLC39A10^{b} | Rabbit | Novusbio | N-terminus, SEQ ID NO: 14 36-85 |
| antibody | polyclonal | | LHRQHRGMTELEPSKFSKQAAENEKKYYIEKL |
| | | | FERYGENGRLSFFGLEKL |

### REFERENCES

1. Knowlden, J. M. et al. Elevated levels of epidermal growth factor receptor/c-erbB2 heterodimers mediate an autocrine growth regulatory pathway in tamoxifen-resistant MCF-7 cells. Endocrinology 144, 1032-1044 (2003).
2. Taylor, K. M. et al. ZIP7-mediated intracellular zinc transport contributes to aberrant growth factor signaling in antihormone-resistant breast cancer Cells. Endocrinology 149, 4912-4920 (2008).
3. Geiser, J., Venken, K. J., De Lisle, R. C. & Andrews, G. K. A mouse model of acrodermatitis enteropathica: loss of intestine zinc transporter ZIP4 (S1c39a4) disrupts the stem cell niche and intestine integrity. PLoS genetics 8, e1002766 (2012).
5. Taylor, K. M., Morgan, H. E., Johnson, A., Hadley, L. J. & Nicholson, R. I. Structure-function analysis of LIV-1, the breast cancer-associated protein that belongs to a new subfamily of zinc transporters. The Biochemical journal 375, 51-59 (2003).
6. Taylor, K. M., Morgan, H. E., Johnson, A. & Nicholson, R. I. Structure-function analysis of HKE4, a member of the new LIV-1 subfamily of zinc transporters. The Biochemical journal 377, 131-139 (2004).
7. Hogstrand, C., Kille, P., Ackland, M. L., Hiscox, S. & Taylor, K. M. A mechanism for epithelial-mesenchymal transition and anoikis resistance in breast cancer triggered by zinc channel ZIP6 and STAT3 (signal transducer and activator of transcription 3). The Biochemical journal 455, 229-237 (2013).
8. Taylor, K. M., Hiscox, S., Nicholson, R. I., Hogstrand, C. & Kille, P. Protein kinase CK2 triggers cytosolic zinc signaling pathways by phosphorylation of zinc channel ZIP7. Science signaling 5, rail (2012).

1. A method of treating or preventing a hyper proliferative disorder by preventing mitosis comprising:
administering a therapeutically effective amount of at least one antibody, or a fragment thereof, that selectively binds the extracellular domain of at least one (Zrt, Irt-like Protein (ZIP) transporter and inhibits function of said ZIP transporter, wherein said ZIP transporter is ZIP6, ZIP10 or a heterodimer comprising both, wherein said at least one antibody or a fragment thereof binds between N terminal amino acids 93-350 for ZIP6 and 46-395 for ZIP10.

2. The method according to claim 1, wherein said fragment comprises at least the Complementarity Determining Region(s) of said at least one antibody.

3. The method of claim 1, wherein said at least one antibody binds the extracellular domains of said ZIP6 and/or ZIP10 transporter, between: N-terminus 1-325 or 1-350 for ZIP6 and 1-407 for ZIP10; or between N-terminus 31-325 or 31-350 for ZIP6 and 31-407 for ZIP10.

4. The method of claim 1, wherein said at least one antibody binds between N terminal amino acids 220-350 for ZIP6 and 46-395 for ZIP10.

5. The method of claim 1, wherein said at least one antibody binds within one of the following extracellular domains (ECDs) of ZIP6:
N-terminus epitope
(SEQ ID NO: 1 93-HHDHDHHSDHEHHSD-107);
N-terminus epitope
(SEQ ID NO: 2 246-EPRKGFMYSRNTNE-259);
N-terminus and including Transmembrane domain 1
(SEQ ID NO: 3 301-RSCLIHTSEK
KAEIPPKTYS LQIAWVGGFI AISIISFLSL LGVILVPLMN-350);
N-terminus
(SEQ ID NO: 4 303-CLIHTSEKKAEIP-315);
N-terminus,
(SEQ ID NO: 5 129-
HSHHNHAASGKNKRKALCPDHDSDSSGKDPRNSQGKGAHRPEHASGRRNV
KDSVSASEVTSTVYNTVSEGTHFLETIETPRPGKLFPKDVSSSTPPSVTS
KSRVSRLAGRKTNESVSEPRKGFMYSRNTNENPQE-263);
or
N-terminus,
(SEQ ID NO: 6 288-NYLCPAIINQIDARSCLIHTSEKKAEIPPKTY
SLQIAWVGGFIAISIISF-337);
or
within one of the following ECDs of ZIP10:
N-terminus,
(SEQ ID NO: 11 46-LEPSKFSKQAAENE-59);
N-terminus
(SEQ ID NO: 12 164-EKETVEVSVKSDDKHMHDHNHRLRHHHRL
HHHLDHNTHHFHNDSITPSERGEPSNEPSTETNKTQEQSDVKLPKGKR
KKKGRKSNENSEVITPGFP-253);
N-terminus,
(SEQ ID NO: 13 295-QDLDPDNEGELRHTRKREAPHVKNNAIIS
LR-395);
or
N-terminus,
(SEQ ID NO: 14 36-LHRQHRGMTELEPSKFSKQAAENEKKYYIE
KLFERYGENGRLSFFGLEKL-85).

6. The method of claim 1, wherein said at least one antibody is selected from group consisting of: ZIP6M, ZIP6Y, ZIP6AM, ZIP6X, ZIP6R, Anti-SLC39A6^{a}, Anti-SLC39A6^{b}, Anti-SLC39A6^{c}, Anti-SLC39A6^{d}, LIV-1/ZIP6 antibody, Anti-SLC39A6^{e}, Anti-SLC39A6^{f}, ZIP62-A, ZIP10, Anti-SLC39A10^{a}, Anti-SLC39A10^{b}, SLC39A10^{a}, Anti-SLC39A10^{c} and SLC39A10^{b}.

7. The method of claim 1, wherein said at least one antibody is selected from the group comprising: ZIP6Y, ZIP6AM, ZIP10 and Anti-SLC39A10^{a}.

8. The method of claim 1, wherein said method comprises at least two antibodies, or fragments thereof, that are used in combination.

9. The method of claim 1, wherein said method comprises at least one antibody that binds at least a part of the ZIP6 transporter and at least one antibody that binds at least a part of the ZIP10 transporter.

10. The method of claim 1, wherein said method comprises a single antibody that binds the ZIP6:ZIP10 heterodimer that inhibits function of the ZIP6:ZIP10 heterodimer.

11. The method of claim 1, wherein said hyper proliferative disorder is selected from the group consisting of: cancer; polycystic kidney disease (PKD) and related cystic kidney diseases; melanoma; hyperplasia, metaplasia and dysplasia's and their various forms; proliferative disorders of the immune system (such as myelo- and lymphoproliferative disorders); prostatic hypertrophy; endometriosis; psoriasis; tissue repair and aberrant wound healing; and fibrosis such as pulmonary fibrosis, idiopathic pulmonary fibrosis, cirrhosis, endomyocardial fibrosis, vascular or spinal stenosis, mediastinal fibrosis, myelofibrosis, retroperitoneal fibrosis, progressive massive fibrosis, nephrogenic systemic fibrosis, Crohn's Disease, keloid or old myocardial infarction, scleroderma/systemic sclerosis, arthrofibrosis, and adhesive capsulitis.

12. The method of claim 11, wherein the cancer is selected from the group consisting of: solid tumours, lymphoma, leukemia, breast, prostate, colon, brain, lung, pancreatic, gastric, bladder, oesophageal and kidney cancer.

13. A pharmaceutical or veterinary composition, comprising: at least one antibody, or a fragment thereof, that selectively binds the extracellular domain of at least one ZIP transporter and inhibits function of said ZIP transporter, wherein said ZIP transporter is ZIP6, ZIP10 or a heterodimer comprising both, wherein said at least one antibody or a fragment thereof binds between N terminal amino acids 93-350 for ZIP6 and 46-395 for ZIP10; and
a pharmaceutically or veterinary acceptable excipient or carrier.

14. A combination therapeutic, comprising:
at least one antibody, or a fragment thereof, that selectively binds the extracellular domain of at least one ZIP transporter and inhibits function of said ZIP transporter, wherein said ZIP transporter is ZIP6, ZIP10 or a heterodimer comprising both, wherein said at least one antibody or a fragment thereof binds between N terminal amino acids 93-350 for ZIP6 and 46-395 for ZIP10; and
at least one other therapeutic agent.

15. An antibody, or fragment thereof, that is specific for ZIP10 wherein said antibody is specific for epitope N-terminus 46-59 amino acids SEQ ID NO: 11 LEPSKFSKQAAENE of the ZIP10 transporter.

16. The pharmaceutical or veterinary composition according to claim 13, comprising: at least one antibody, or a fragment thereof, that selectively binds the extracellular domain of at least one ZIP transporter and inhibits function of said ZIP transporter, wherein said ZIP transporter is ZIP6, ZIP10 or a heterodimer comprising both, wherein said at least one antibody or a fragment thereof binds between N terminal amino acids binds between N terminal amino acids 220-350 for ZIP6 and 46-395 for ZIP10.

17. The combination therapeutic according to claim 14, comprising:
at least one antibody, or a fragment thereof, that selectively binds the extracellular domain of at least one ZIP transporter and inhibits function of said ZIP transporter, wherein said ZIP transporter is ZIP6, ZIP10 or a heterodimer comprising both, wherein said at least one antibody or a fragment thereof binds between N terminal amino acids binds between N terminal amino acids 220-350 for ZIP6 and 46-395 for ZIP10; and
at least one other therapeutic agent.


SEQUENCE LISTING (23 sequences)

SEQ ID NO: 1 - protein, 15 aa, Homo sapiens
HHDHDHHSDHEHHSD

SEQ ID NO: 2 - protein, 14 aa, Homo sapiens
EPRKGFMYSRNTNE

SEQ ID NO: 3 - protein, 50 aa, Homo sapiens
RSCLIHTSEKKAEIPPKTYSLQIAWVGGFIAISIISFLSLLGVILVPLMN

SEQ ID NO: 4 - protein, 13 aa, Homo sapiens
CLIHTSEKKAEIP

SEQ ID NO: 5 - protein, 135 aa, Homo sapiens
HSHHNHAASGKNKRKALCPDHDSDSSGKDPRNSQGKGAHRPEHASGRRNVKDSVSASEVTSTVYNTVSEGTHFLETIETPRPGKLFPKDVSSSTPPSVTSKSRVSRLAGRKTNESVSEPRKGFMYSRNTNENPQE

SEQ ID NO: 6 - protein, 50 aa, Homo sapiens
NYLCPAIINQIDARSCLIHTSEKKAEIPPKTYSLQIAWVGGFIAISIISF

SEQ ID NO: 7 - protein, 41 aa, Homo sapiens
ASHHHSHSHEEPAMEMKRGPLFSHLSSQNIEESAYFDSTWK

SEQ ID NO: 8 - protein, 7 aa, Homo sapiens
TEGLSSG

SEQ ID NO: 9 - protein, 8 aa, Homo sapiens
HYAENVSM

SEQ ID NO: 10 - protein, 8 aa, Homo sapiens
KIVFRINF

SEQ ID NO: 11 - protein, 14 aa, Homo sapiens
LEPSKFSKQAAENE

SEQ ID NO: 12 - protein, 96 aa, Homo sapiens
EKETVEVSVKSDDKHMHDHNHRLRHHHRLHHHLDHNTHHFHNDSITPSERGEPSNEPSTETNKTQEQSDVKLPKGKRKKKGRKSNENSEVITPGFP

SEQ ID NO: 13 - protein, 31 aa, Homo sapiens
QDLDPDNEGELRHTRKREAPHVKNNAIISLR

SEQ ID NO: 14 - protein, 50 aa, Homo sapiens
LHRQHRGMTELEPSKFSKQAAENEKKYYIEKLFERYGENGRLSFFGLEKL

SEQ ID NO: 15 - protein, 33 aa, Homo sapiens
GGHDHSHQHAHGHGHSHGHESNKFLEEYDAVLK

SEQ ID NO: 16 - protein, 7 aa, Homo sapiens
SAGLTGG

SEQ ID NO: 17 - protein, 10 aa, Homo sapiens
QYANNITLWN

SEQ ID NO: 18 - protein, 8 aa, Homo sapiens
KIVFDIQF

SEQ ID NO: 19 - protein, 755 aa, Homo sapiens
MARKLSVILILTFALSVTNPLHELKAAAFPQTTEKISPNWESGINVDLAISTRQYHLQQLFYRYGENNSLSVEGFRKLLQNIGIDKIKRIHIHHDHDHHSDHEHHSDHERHSDHEHHSEHEHHSDHDHHSHHNHAASGKNKRKALCPDHDSDSSGKDPRNSQGKGAHRPEHASGRRNVKDSVSASEVTSTVYNTVSEGTHFLETIETPRPGKLFPKDVSSSTPPSVTSKSRVSRLAGRKTNESVSEPRKGFMYSRNTNENPQECFNASKLLTSHGMGIQVPLNATEFNYLCPAIINQIDARSCLIHTSEKKAEIPPKTYSLQIAWVGGFIAISIISFLSLLGVILVPLMNRVFFKFLLSFLVALAVGTLSGDAFLHLLPHSHASHHHSHSHEEPAMEMKRGPLFSHLSSQNIEESAYFDSTWKGLTALGGLYFMFLVEHVLTLIKQFKDKKKKNQKKPENDDDVEIKKQLSKYESQLSTNEEKVDTDDRTEGYLRADSQEPSHFDSQQPAVLEEEEVMIAHAHPQEVYNEYVPRGCKNKCHSHFHDTLGQSDDLIHHHHDYHHILHHHHHQNHHPHSHSQRYSREELKDAGVATLAWMVIMGDGLHNFSDGLAIGAAFTEGLSSGLSTSVAVFCHELPHELGDFAVLLKAGMTVKQAVLYNALSAMLAYLGMATGIFIGHYAENVSMWIFALTAGLFMYVALVDMVPEMLHNDASDHGCSRWGYFFLQNAGMLLGFGIMLLISIFEHKIVFRINF

SEQ ID NO: 20 - protein, 831 aa, Homo sapiens
MKVHMHTKFCLICLLTFIFHHCNHCHEEHDHGPEALHRQHRGMTELEPSKFSKQAAENEKKYYIEKLFERYGENGRLSFFGLEKLLTNLGLGERKVVEINHEDLGHDHVSHLDILAVQEGKHFHSHNHQHSHNHLNSENQTVTSVSTKRNHKCDPEKETVEVSVKSDDKHMHDHNHRLRHHHRLHHHLDHNNTHHFHNDSITPSERGEPSNEPSTETNKTQEQSDVKLPKGKRKKKGRKSNENSEVITPGFPPNHDQGEQYEHNRVHKPDRVHNPGHSHVHLPERNGHDPGRGHQDLDPDNEGELRHTRKREAPHVKNNAIISLRKDLNEDDHHHECLNVTQLLKYYGHGANSPISTDLFTYLCPALLYQIDSRLCIEHFDKLLVEDINKDKNLVPEDEANIGASAWICGIISITVISLLSLLGVILVPIINQGCFKFLLTFLVALAVGTMSGDALLHLLPHSQGGHDHSHQHAHGHGHSHGHESNKFLEEYDAVLKGLVALGGIYLLFIIEHCIRMFKHYKQQRGKQKWFMKQNTEESTIGRKLSDHKLNNTPDSDWLQLKPLAGTDDSVVSEDRLNETELTDLEGQQESPPKNYLCIEEEKIIDHSHSDGLHTIHEHDLHAAAHNHHGENKTVLRKHNHQWHHKHSHHSHGPCHSGSDLKETGIANIAWMVIMGDGIHNFSDGLAIGAAFSAGLTGGISTSIAVFCHELPHELGDFAVLLKAGMTVKQAIVYNLLSAMMAYIGMLIGTAVGQYANNITLWIFAVTAGMFLYVALVDMLPEMLHGDGDNEEHGFCPVGQFILQNLGLLFGFAIMLVIALYEDKIVFDIQF

SEQ ID NO: 21 - protein, 20 aa, Callithrix jacchus
DIKKQLSKYESQLSANEEKV

SEQ ID NO: 22 - protein, 20 aa, Mus musculus
ESKKQLSKYDSQLSSNEEKV

SEQ ID NO: 23 - protein, 20 aa, Rattus norvegicus
ESKKQLSKYESQLSTNEEKV